(19) United States
(12) Patent Application Publication
(10) Pub. No.: US 2025/0261989 A1
(43) Pub. Date: Aug. 21, 2025

(54) NASAL AIRWAY TISSUE TREATMENT SYSTEM AND METHOD

(71) Applicant: Aerin Medical Inc., Mountain View, CA (US)

(72) Inventors: Scott J. Wolf, Menlo Park, CA (US); Andrew Frazier, Sunnyvale, CA (US); Christopher James Foster, San Francisco, CA (US)

(73) Assignee: Aerin Medical Inc., Mountain View, CA (US)

(21) Appl. No.: 19/057,852

(22) Filed: Feb. 19, 2025

Related U.S. Application Data
(60) Provisional application No. 63/555,150, filed on Feb. 19, 2024.

(51) Int. Cl. A61B 18/14; A61B 17/00; A61B 18/00

(52) U.S. Cl. CPC A61B 18/1485; A61B 2017/00893; A61B 2018/00327; A61B 2018/00434; A61B 2018/00577; A61B 2018/00696; A61B 2018/00708; A61B 2018/00821; A61B 2018/00875; A61B 2018/1467

(57) ABSTRACT

Systems and methods are provided for treating tissue in a nasal airway. In some embodiments, a console including a pulsed electric field generator is provided to generate pulsed electric field energy in the form of high frequency alternating pulses of energy delivered as a pulse train. The pulsed electric field generator including a plurality of output channels. The system also includes a treatment device including an elongate shaft having a treatment surface on a distal end thereof, the treatment surface including a plurality of electrode pairs configured to deliver the pulsed electric field energy provided by the pulsed electric field generator to a target tissue in the nasal airway. Each of the plurality of output channels is connected with a subset of the plurality of electrode pairs to provide pulsed electric field energy to the plurality of electrode pairs.

### RELATED APPLICATIONS

This application claims the benefit of and priority to U.S. Provisional Application No. 63/555,150 filed Feb. 19, 2024, the contents of which is hereby incorporated herein by reference in its entirety.

### FIELD

The present application is related to medical devices and methods, and more specifically, relates to a system and method for treating nasal airway tissue using pulsed field energy.

### BACKGROUND

Rhinitis, or inflammation and swelling of the mucous membrane of the nose, causes symptoms such as runny nose, itchy nose, and congestion. Chronic and allergic rhinitis affect tens of millions of patients every year in the United States alone-one of the most common reasons for patients to visit physicians. Typically, rhinitis is treated with medications, such as nasal sprays and allergy shots. Unfortunately, these medications are temporary and are associated with multiple side effects. Surgical options are limited and invasive, typically involving removal of tissue in the nasal cavity.

The human nasal cavity is an area that extends from inside the two nostrils at the front of the nose to the junction of the nasal cavity with the back of the throat. The nasal septum is a wall of cartilage and bone that separates an anterior portion of the nasal cavity into the two nostrils. The lateral walls and septum of the nasal cavity are formed mainly of cartilage and bone, covered with a mucous membrane (or “nasal mucosa”). Other tissues also reside below the nasal mucosa, such as small blood vessels and nerves. Tissue underlying the nasal mucosa is sometimes referred to generally as submucosal tissue. One of the functions of mucosal tissue is to produce mucus, which helps protect, moisten and clear debris from the nasal cavities. Nerves underlying the nasal mucosa are generally responsible for sensory function—feeling and smelling—and for activating the mucus-producing cells in the nasal mucosa to produce mucus.

Various treatments for rhinitis have been used but have many drawbacks and side effects. For example, turbinate reduction surgery is one surgical treatment for chronic or allergic rhinitis but is relatively invasive with only temporary effects and can result in complications, such as mucosal sloughing, acute pain and swelling, and bone damage. Transection of the vidian nerve was also used, but can result in side effects including dry eye, because autonomic fibers in the vidian nerve innervate the lacrimal glands.

Another surgical treatment method for chronic or allergic rhinitis, posterior nasal neurectomy, was initially developed in the late 1990s. Posterior nasal neurectomy involves cutting or cauterizing nerve tissue located in or near the sphenopalatine foramen, an opening in each of the two the lateral walls of the nasal cavity, where the sphenopalatine ganglion is located and out of which multiple nerves branch. This procedure was difficult due to the location posterior location of the nasal cavity and destroying nerves in that area may result in unwanted side effects.

Therefore, it would be desirable to have improved devices, systems and methods for treating chronic and allergic rhinitis. Ideally, such devices, systems and methods would have longer term effects than medications and be less invasive than currently available surgical techniques.

### BRIEF SUMMARY

In some embodiments, a system is provided for treating tissue in a nasal airway. The system includes a console including a pulsed electric field generator configured to generate pulsed electric field energy in the form of high frequency alternating pulses of energy delivered as a pulse train. The pulsed electric field generator includes a plurality of output channels. The system also includes a treatment device including an elongate shaft having a treatment surface on a distal end thereof, the treatment surface including a plurality of electrode pairs configured to deliver the pulsed electric field energy provided by the pulsed electric field generator to a target tissue in the nasal airway. Each of the plurality of output channels is connected with a subset of the plurality of electrode pairs to provide pulsed electric field energy to the plurality of electrode pairs.

In some embodiments, the pulsed electric field generator is configured to deliver pulsed field ablation energy to the plurality of electrode pairs. In some embodiments, the pulsed electric field generator is configured to deliver pulsed radiofrequency energy to the plurality of electrode pairs. In some embodiments, the pulse train is configured to be delivered in repeated intervals to create a string of pulse trains with an interval of time between pulse trains to allow for tissue recovery between each pulse train. In some embodiments, the pulsed electric field generator is configured to provide at least one of exponentially decaying, square wave, bipolar, or oscillatory pulses.

In some embodiments, the plurality of output channels is configured to connect with the plurality of electrode pairs to provide independent pulsed electric energy from each of the plurality of output channels to each of subsets of the plurality of electrode pairs.

In some embodiments, the system can further include at least one sensor configured to measure a temperature of the target tissue. In some embodiments, the shaft of the treatment device is malleable and has a width of 4 millimeters to 5 millimeters. In some embodiments, the plurality of electrode pairs includes at least two rows of electrodes pairs.

In some embodiments, a system is provided for treating nasal airway tissue. The system includes a pulsed electric field generator configured to generate pulsed electric field energy in the form of alternating pulses of energy delivered as a pulse train. The pulsed electric field generator includes a plurality of output channels. The system also includes a treatment device including an elongate shaft having a treatment surface on a distal end thereof. The treatment surface includes a first row of electrodes including a first plurality of electrode pairs and a second row of electrodes including a second plurality of electrode pairs configured to deliver pulsed electric field energy provided by the pulsed electric field generator to a target tissue in the nasal airway. Each of the plurality of output channels is connected with a subset of the first and second plurality of electrode pairs to provide pulsed electric field energy to the plurality of electrode pairs.

In some embodiments, a method is provided for treating a nasal airway that includes activating a pulsed electric field generator console attached to a treatment device to generate pulsed electric field energy in the form of high frequency alternating pulses of energy delivered as a pulse train, advancing a distal tip of the treatment device into the nasal airway of a patient, contacting a nasal mucosa lining the nasal airway with a treatment surface at a distal end of the treatment device, delivering the pulsed electric field energy from at least a first electrode on the treatment surface to at least a second electrode on the treatment surface to treat a tissue underlying the nasal mucosa lining, and removing the distal tip of the treatment device from the nasal airway.

In some embodiments, the method further includes moving the distal tip to multiple additional locations within the nasal airway; and delivering the pulsed electric field energy to nasal airway tissue at the multiple additional locations. In some embodiments, the tissue includes at least one nasal nerve selected from the group consisting of a posterior nasal nerve and an anterior ethmoid nerve.

In some embodiments, the method, further includes sensing a temperature of the nasal mucosa lining with a temperature sensing member on the treatment surface of the distal tip. In some embodiments, the method further includes automatically shutting off delivery of the pulsed electric field energy from the pulsed electric field generator if the temperature is above a predefined acceptable maximum temperature.

In some embodiments, the method further includes sensing an impedance in the tissue between the first electrode and the second electrode with an impedance sensing member; and automatically shutting off delivery of the pulsed electric field energy from the pulsed electric field generator console to the treatment device if the impedance is outside a predefined acceptable impedance range.

In some embodiments, the at least the first electrode includes a first row of electrodes, and wherein the at least the second electrode includes a second row of electrodes.

In some embodiments, the method further includes injecting an anesthetic fluid into the nasal mucosa lining before advancing the distal tip of the treatment device into the nasal airway, to enhance conduction of the pulsed electric field energy through the tissue.

In some embodiments, delivering the pulsed electric field energy ablates the tissue underlying the nasal mucosa lining. In some embodiments, the tissue includes nerve tissue, and the method further includes delivering the pulsed electric field energy to an additional tissue selected from the group consisting of an inferior turbinate, a middle turbinate, a superior turbinate, a nasal septum, and a septal swell body.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 illustrates an exemplary embodiment of a system for treating nasal airway tissue;

FIGS. 2A and 2B illustrate exemplary embodiments of a system for treating tissue having a plurality of output channel for delivering energy to tissue;

FIG. 3 is a front perspective view of an electrosurgical system for treating nasal airway tissue, according to one embodiment;

FIG. 4 is a perspective view of the console of FIG. 3, shown connected to an on/off foot pedal and a power cord, according to one embodiment;

FIG. 5 is a perspective view of an electrosurgical system, according to an alternative embodiment;

FIG. 6 is a rear perspective view of the console of the electrosurgical system of FIG. 5;

FIGS. 7A, 7B, and 7C are top, front and side views, respectively, of the console shown in FIGS. 5 and 6;

FIG. 8 is a screen shot of a standby screen display that may be displayed on an electrosurgical console, according to one embodiment;

FIG. 9 is a screen shot of a default main screen display on an electrosurgical console, as it appears before the electrosurgery treatment has started, according to one embodiment;

FIG. 10 is a screen shot of a default main screen display on an electrosurgical console, as it appears during an RF delivery stage of an electrosurgery treatment, according to one embodiment;

FIG. 11 is a screen shot of a default main screen display on an electrosurgical console, as it appears during a cooling stage of an electrosurgery treatment, according to one embodiment;

FIG. 12 is a screen shot of a custom treatment settings screen display on an electrosurgical console, as it appears during a cooling stage of an electrosurgery treatment, according to one embodiment;

FIG. 13 is a screen shot of a fault screen display on an electrosurgical console, according to one embodiment;

FIG. 14 is a screen shot of an error screen display on an electrosurgical console, according to one embodiment;

FIG. 15 is a screen shot of a settings screen display on an electrosurgical console, according to one embodiment;

FIG. 16 is a flow diagram of a method of performing an electrosurgical treatment, using a console as described in the present application, according to one embodiment;

FIG. 17 is a close-up perspective view of the stylus, cable and connector of the system of FIG. 3;

FIGS. 18A and 18B are exemplary embodiments of side and top perspective views, respectively, of a distal tip (or “treatment element”) of a treatment device or stylus;

FIGS. 19A and 19B are side and front views, respectively, of a distal end of a nasal airway tissue treatment stylus, according to an alternative embodiment;

FIG. 20 is a front view of a distal end of a nasal airway tissue treatment stylus, according to an alternative embodiment;

FIG. 21 is a flow chart illustrating an exemplary method for treating nasal airway tissue using the systems described herein;

FIG. 22 is a top view of the stylus of FIGS. 3 and 17-18B, illustrating a method for bending the shaft of the stylus, according to one embodiment;

FIGS. 23A, 23B, and 23C illustrate a method of using a shaft bending tool to bend a stylus, according to one embodiment;

FIGS. 24A, 24B, and 24C illustrate a method of using a different shaft bending tool to bend a stylus, according to an alternative embodiment;

FIG. 25 is a perspective view of a nasal airway tissue treatment stylus with two bends in the shaft and bend markers on the shaft, according to one embodiment;

FIG. 26 is a perspective view of the stylus of FIGS. 3, 17-18B, and 22, illustrating an indicator on the handle showing the direction of treatment, according to one embodiment;

FIG. 27 is a sagittal view of a human nasal cavity, illustrating a method for treating the posterior nasal nerve with the stylus described herein, according to one embodiment;

FIGS. 28A and 28B illustrate a method for addressing, contacting and treating nasal tissue, according to one embodiment;

FIG. 29 illustrates an improper treatment contact with nasal mucosa;

FIGS. 30A and 30B are perspective and front views, respectively, of an alternative embodiment of a nasal airway tissue treatment stylus that includes an expandable wire electrode component;

FIGS. 30C and 30D are perspective views of the stylus of FIGS. 30A and 30B, illustrating operation of the expandable wire electrode component;

FIG. 31 is perspective view of another alternative embodiment of a nasal airway tissue treatment stylus that includes an expandable cryotherapy balloon;

FIGS. 32A and 32B are perspective and front views, respectively, of another alternative embodiment of a nasal airway tissue treatment stylus that includes an expandable wire electrode component that advances out of the distal tip of the device;

FIGS. 33A, 33B, and 33C illustrate exemplary embodiments of a treatment device having an expandable wire electrode component;

FIGS. 34 and 35 illustrate exemplary embodiments of a treatment device having a multi-electrode treatment element in the form of a basket;

FIGS. 36A, 36B, and 36C illustrate exemplary embodiments of multi-channel electrodes;

FIGS. 37A, 37B, 37C, 37D, 37E, 37F, and 37G illustrate exemplary graphs showing biphasic and monophasic pulses;

FIGS. 38, 39A, 39B, and 40 illustrate various embodiments of circuits for producing the pulsed energy;

FIGS. 41A and 41B illustrate the mechanism of electroporation and cell death that can be achieved using PFA;

FIG. 42 illustrates an exemplary flow diagram of an example method of use of the various devices disclosed herein;

FIGS. 43A and 43B show an exemplary cross-sectional view of the mucosa;

FIGS. 44A and 44B show sagittal views of a human nasal cavity;

FIGS. 45A, 45B, 45C, and 45D illustrate anatomical elements of a human nose; and

FIG. 45E is a sagittal view of a human nasal cavity, illustrating the lateral wall and nasal nerves.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following disclosure provides embodiments of systems and methods for treating nasal conditions, treating migraines, and/or improving breathing by decreasing airflow resistance or perceived airflow resistance at or near a site of an internal or external nasal valve. Such embodiments may include methods and devices for reshaping, remodeling, strengthening, or changing the properties of the tissues of the nose, including, but not limited to the skin, muscle, mucosa, submucosa, and cartilage in the area of the nasal valves. For example, the devices described herein can be used for nerve destruction for posterior nasal nerve treatment for treatment of chronic rhinitis, treatment of migraines through nasal nerve treatments, or other diseases that can be treated with nerve ablation that are in the ears, nose, or throat.

This application describes various aspects and embodiments of a device, system, and method for treating nasal airway tissue. More particularly, the system includes: (1) a console with an electrosurgical generator and a display; and (2) a treatment device, or stylus, coupled to the console via a cable. In some embodiments, the electrosurgical generator is configured to provide pulsed electric field (PEF) energy for conducing pulsed radiofrequency energy (PRF) or pulsed field ablation (or “PEF ablation”) of one or more tissues. In some embodiments, the electrosurgical generator is configured to provide radiofrequency (RF) energy and can be delivered continuously or through short duration pulses to minimize the thermal effect on adjacent tissue. In some embodiments, the electrosurgical generator is configured to provide high-voltage direct current pulses of short duration to induce regulated cell death (e.g. apoptosis) through irreversible electroporation, this treatment modality has minimal thermal effect and can target specific tissues while minimizing collateral damage. In alternative embodiments, the system may be configured to deliver another type of energy, such as but not limited to heat, laser, microwave, cryogenic cooling, DC current or ultrasound.

The energy delivery system may be designed and used to perform a number of different types of nasal airway tissue treatments. For example, and as further described in the Incorporated References, in some embodiments, the system may be used to reshape, reconfigure and/or change another property of tissue (such as but not limited to cartilage) in or near a nasal valve area within the nose to enhance nasal breathing. In another embodiment, the system may be used to treat soft tissue in an anterior and/or posterior portion of the nasal airway to treat chronic rhinitis, allergic rhinitis, post nasal drip and/or chronic cough. Tissues treated in such a procedure may include nasal nerves (posterior nasal nerve, vidian nerve branches, sphenopalatine ganglion, anterior ethmoid nerve, etc.), submucosal tissue, mucosal tissue, goblet cells and/or the like. Although the description focuses primarily on treating nerves in the nasal cavity to treat chronic rhinitis, allergic rhinitis, post nasal drip and/or chronic cough, the methods, systems and devices described herein may alternatively be used to perform any suitable procedure in a nasal airway, including but not limited to all procedures described in the Incorporated References.

The systems disclosed herein can utilize various types of energy, including pulse radiofrequency energy and pulse field ablation energy, to affect the nasal tissue. Pulsed radiofrequency energy technology can be used to deliver electrical energy to modulate or destroy nerves, specifically the posterior nasal nerve. Due to the non-thermal nature of the technology, adjacent tissue damage is minimized (and heals effectively). In some embodiments, the system delivers at least one pulse train at the treatment site to cause irreversible electroporation of the nerve tissue, which induces regulated cell death. The treatments can be delivered using any of the devices described here for precise targeting of the nasal nerves which are located submucosally and run along the middle meatus, middle turbinate, inferior turbinate, and inferior meatus (in addition to nerves located inside the nasal cavity). Pulsed field ablation (PFA) technology can be used to deliver energy to cause cell death, for example, through the use of high voltage pulsing that induce regulated cell death, or apoptosis, through irreversible electroporation of targeted cells.

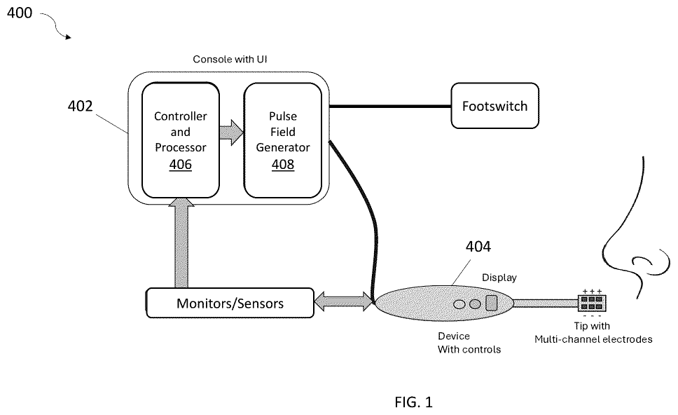

FIG. 1 illustrates an exemplary embodiment of a nasal airway treatment system 400 that includes a console 402 for controlling energy delivery to a plurality of pairs of electrodes on a treatment device 404 or stylus and displaying information to the user. The console includes a controller 406 and an energy generator 408. In some embodiments, the energy generator is a pulsed field generator for producing pulsed energy, such as PFA or PRF.

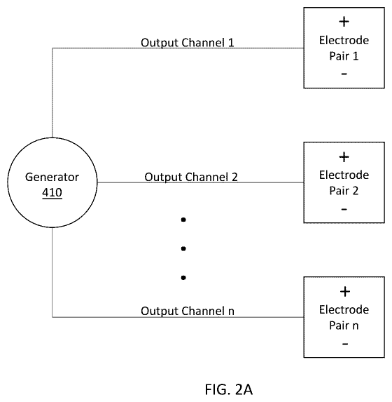

In some embodiments, the console includes one or more ports, or output channels, for connecting to the treatment device or stylus, for treating one or more target tissues in the nasal cavity with energy. In some embodiments, each pair of electrodes on the treatment device can be connected to a separate, controlled electrical output channel to allow for different electrode pairs of the treatment element to be activated separately and independently of one another, as shown in FIGS. 2A and 2B. For example, FIG. 2A illustrates a generator 410 that comprises output channel 1 through output channel n for delivering energy to electrodes. For example, FIG. 2B illustrates a console that includes a plurality of output channels 420 for delivering energy. By controlling the treatment energy flowing through each pair of electrodes using parameters including, but not limited to, temperature, a greater degree of control and accuracy over the treatment energy may be obtained, such that treatments may be repeatable to each electrode pairs or subsets of electrode pairs. In some embodiments, each of the plurality of output channels can be assigned to an individual electrode pair. In some embodiments, each of the plurality of output channels can be assigned to a subset of the plurality of electrode pairs.

More specifically, in some embodiments of treatment devices comprising an array or multiple pairs of electrodes, each pair of electrodes (bipolar) or each electrode (monopolar) may have a separate, controlled electrical output channel to allow for different regions of the treatment element to be activated separately.

The output channels may also comprise separate or integrated feedback. This may allow for more accurate temperature control and more precise targeting of tissue. Separate control may also allow energy to be focused and/or intensified on a desired region of the treatment element in cases where the anatomy of the nasal tissue/structures does not allow the entire electrode region of the treatment element to engage the tissue. In such embodiments, the nasal tissue that is in contact with the treatment element may receive sufficient energy to treat the tissue.

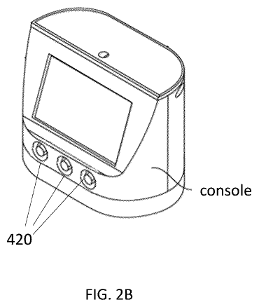

Referring to FIG. 3, in some embodiments, an exemplary embodiment of a nasal airway tissue treatment system 10 includes two primary components: a console 12 and a treatment device or stylus 20. Similar to the console shown above, the console 12 includes a display 14, an electrosurgical generator, and electronics (inside the console 12 and thus not visible), and an outlet 16 into which the stylus 20 is plugged. The stylus 20 includes a handle 22, a shaft 24, a distal tip 26 (also referred to as a “treatment element”), a cable 28 and a connector 29 for connecting the stylus 20 with the outlet 16. Many features of the console 12 and the system 10 in general are described further below, in U.S. patent application Ser. No. 16/668,678, and in the other Incorporated References. In some embodiments, the electrosurgical generator is configured to provide pulsed electric field (PEF) energy for conducing pulsed field ablation (or “PEF ablation”) of one or more tissues, including but not limited to any suitable nerve tissue.

The console 12 is a reusable device, which is designed and intended for use with multiple patients. The stylus 20, on the other hand, is a single-patient, single-use, disposable device. In some embodiments, the stylus 20 may be provided as part of a stylus kit 70, which may include a curved anesthesia needle 72, a packet of conductive gel 73, a shaft bending tool 74 and/or instructions for use 76 (or “IFU”). Anesthesia protocols for anesthetizing the patient's nasal cavity are largely up to the physician, and many different protocols are known to otolaryngologists. In some embodiments, it may be required or strongly recommended to inject anesthetic into the mucosa and/or submucosa in the target area(s), in order to help direct the current delivered by the stylus 20. This may be helpful in some embodiments, because fluid such as anesthetic is generally conductive for RF. All of these kit components are optional, and any embodiment of the stylus kit 70 may include fewer items or additional items, without departing from the scope of the invention. In some embodiments, the stylus 20 may be provided by itself for use with the console 12. The stylus 20 may be used for multiple treatments on the same patient at the same time—for example multiple treatment areas in a nostril and/or treatment of both nostrils—and then is disposed of after use on that patient. In an alternative embodiment, the stylus 20 may be sterilizable and reusable.

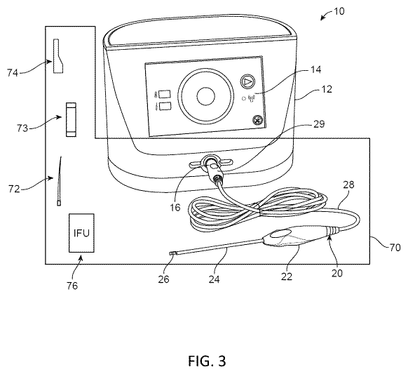

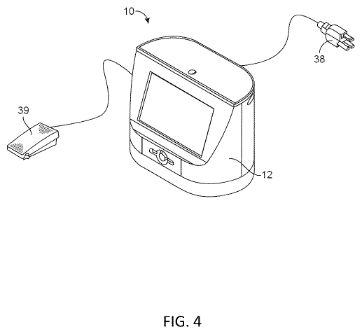

In some embodiments, the nasal airway tissue treatment system 10 may be provided with one or more additional components or accessories. For example, and as shown in FIG. 4, the system 10 typically includes a power cord 38 and a foot switch 39, both of which attach to the console 12. FIG. 4 illustrates the console 12 of the nasal airway tissue treatment system 10, along with a power cord 38 and a foot switch 39, both of which plug into the back of the console 10. The foot switch 39 is used by the physician to activate the stylus 20 during a procedure. In alternative embodiments, the handle 22 of the stylus 20 may include an on/off button or switch, in addition to or as an alternative to the foot switch 39.

As another example, the stylus 20 illustrated in FIG. 3 has a relatively long shaft 24, configured for use in the posterior portion of the nasal cavity to treat nerves to treat rhinitis. In one embodiment, one or more additional styluses may be provided with the system 10, for performing other procedures. For example, a second stylus with a shorter shaft for addressing tissue in the nasal valve, closer to the front of the nose, may be provided. The curved anesthesia needle 72 for injecting anesthetic into the nasal mucosa is another optional component. The curved needle 72 is designed to be stiffer than a spinal needle and is curved to allow a physician to easily access and inject anesthesia into an area near the back of the nasal cavity. The curved needle 72 can also be reinforced to prevent bending or bowing during injection. The system 10 may also be provided with one or more conductive gel packets 73, for application to the distal tip 26 of the stylus, to enhance contact of the distal tip 26 with nasal mucosa. The bending tool 74 will be described further below and may be used by the physician to bend the shaft 24 of the stylus 20 in one or more locations. Any other suitable accessories or components may be added to the treatment system 10, according to alternative embodiments.

According to various embodiments, the console 12 of the treatment system 10 may include default settings and custom settings. Default settings may include, for example, a power output of 4 Watts, a treatment temperature of 60 degrees Celsius, and a treatment time of 12 seconds. Custom settings may allow a physician to customize settings. For example, such settings could provide for power of 3-5 Watts with an increment interval of 1 Watt, a treatment temperature of 50-70 degrees Celsius with an increment interval of 5 degrees Celsius, and a treatment time of 10-12 seconds with an increment interval of 2 seconds. These are merely examples, however, and should not be interpreted as limiting.

Referring to FIG. 5, another example of an electrosurgical system 100 is illustrated. In this embodiment, the stylus 104 has a shorter shaft, such as might be used in treating tissues in the nasal valve area. Thus, the treatment times and protocols, as well as the displays on the display screen 108 of the console 102, are designed for nasal valve treatment. These features may be adjusted/altered for treatment of nasal nerve(s) to treat rhinitis. Similar to the system above, the electrosurgical system 100 can be modified to provide and deliver any suitable type of energy, including pulsed RF and PFA. In this example, the electrosurgical system 100 includes a console 102, an energy delivery stylus 104, and a cable 106 connecting the stylus 104 to the console 102. The console 102 houses an energy generator, a processor and various electronics, none of which is visible in the figure. The word “console,” in this disclosure, is meant to encompass the terms “generator,” “box” and any other commonly used terms to describe an electrosurgical system console or generator. The parts of the console 102 that are visible include a touch screen display 108, a stylus connection port 110, an “Energy ON” indicator light 112, and a bottom ring 114. The touch screen display 108 serves as the main user interface for interacting with the console 102 and will be described further below. The stylus connection port 110 allows a connection end (or “connector”) of the stylus 104 to be plugged into it. The stylus connection port 110 is configured to accept only the connection end of the stylus 104 and will not accept or work with counterfeit or other devices. In some embodiments, electronics inside the console 102 may include a stylus identification safety feature that identifies the stylus 104 when it is plugged into the stylus connection port 110. Such a safety feature may, for example, automatically shut down (or disable powering on) the console 102, if a user tries to plug in a device other than the stylus 104.

The “Energy ON” indicator light 112 indicates when energy is being delivered through the stylus connection port 110 to the stylus 104. The bottom ring 114, in this embodiment, lights up when the console 102 is powered on. This lighted ring 114 is an optional feature. Both the “Energy ON” indicator light 112 and the lighted bottom ring 114 may have any color or colors of light. In one embodiment, for example, the “Energy ON” indicator light 112 is blue, and the bottom ring 114 lights up with a white light. This is merely one example, however, and any suitable lighting configuration and combination of colors may be used in alternative embodiments.

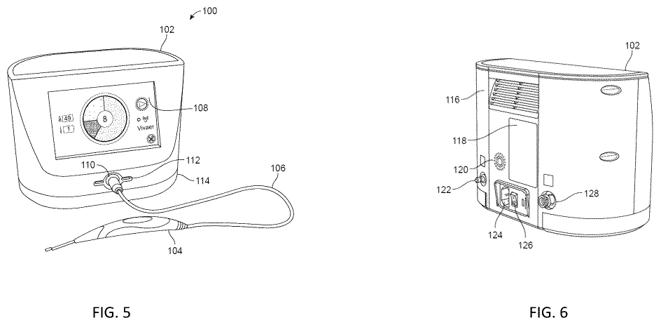

FIG. 6 shows the back of the console 102. In some embodiments, the console 102 includes an air vent 116, a product label area 118, a speaker 120, an equipotential ground connection port 122, a main power connection port 124, a main power switch 126 and foot switch connection port 128. The equipotential ground connection port 122 is provided at the back of the device.

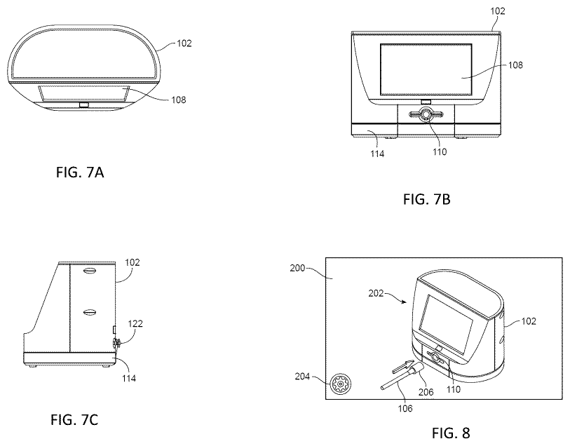

FIGS. 7A, 7B, and 7C are top, front and side views, respectively, of the console 102 shown in FIGS. 5 and 6.

Referring now to FIG. 8, an exemplary screen shot of one example of a standby image 200 that may be shown on the display screen 108 of the console 102 is illustrated. The standby image 200 may be displayed after the console 102 is powered on. In some embodiments, the console 102 may perform a power-on self-test before showing the standby image 200. The standby image includes a console image 202, a settings button 204 and an animated insert stylus image 206. The animated insert stylus image 206 shows the connector end of the stylus cable 106 moving toward the console stylus connection port 110. Thus, the display 200 graphically informs the user that the console 102 is ready for use, awaiting insertion of the connection end of the stylus cable 106 into the connection port 110.

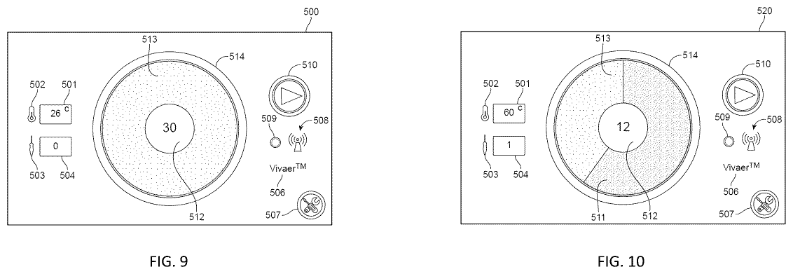

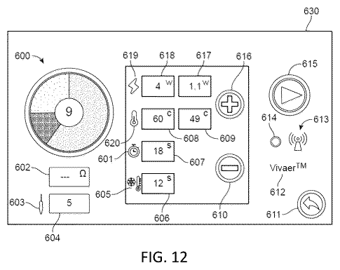

Referring now to FIG. 9, once the physician or other user has attached the stylus 104 to the console 102, the standby image of FIG. 8 may be replaced by a default main screen image 500. This main screen image 500 is what the user sees before the procedure has started. Under normal operating conditions, the user can select either default treatment settings or manual treatment settings. The default treatment settings are pre-loaded into the processor of the console 102 and do not require any additional settings inputs from the user. In some embodiments, it may be possible for the user to select from several sets of default settings. Operation under the default settings mode is described in relation to FIGS. 9, 10, and 11, and operation under the custom settings mode is described in relation to FIG. 12.

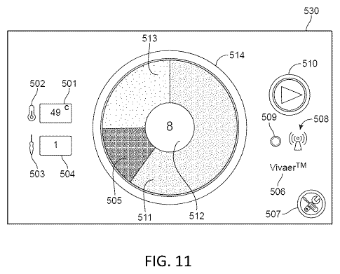

FIG. 10 shows the default image 500 displayed on the display screen 108 of the console 102, once a valid stylus 104 is connected. In this embodiment, the default image includes a stylus temperature indicator 501, a temperature icon 502, a stylus icon 503, treatment number indicator 504, a stylus type connected indicator 506, a start/stop button 510, an RF ON indicator light 509, an energy icon 508, a custom treatment button 507 and a central, circular, graphical treatment progress display 514.

The graphical treatment progress display 514 has several portions, according to the embodiment shown in FIG. 9. First, there is a total treatment timer 512, which is displayed as a central circle with a counting down number, representing seconds (or alternatively minutes, or minutes and seconds) remaining in the current procedure. Here, for example, the treatment has not started yet, and the total treatment timer 512 shows a time of 30 seconds, thus indicating that at least the next tissue treatment will last 30 seconds total. Immediately surrounding the central circle total treatment timer 512 is a treatment time indication ring 513, which acts as an indicator of elapsed and remaining time in the procedure. In the default image 500 of FIG. 9, the treatment has not started, so the entire outer ring 513 is one initial color. The initial color of the ring 513 indicates time remaining in the treatment, which in this case takes up the entire ring 513.

Other indicators on the screen shot image 500 also show that the treatment has not yet started. For example, the “Energy ON” indicator light 509 is not illuminated yet, because the console 102 is not yet delivering energy to the stylus 104. The treatment number indicator 504 shows that zero treatments have been performed with the stylus 104 that is currently plugged into the console 102. And the temperature indicator 501 shows a stylus temperature of 26 degrees Celsius. To begin a treatment, the physician user will touch the start/stop button 510 on the touchscreen 108.

Referring now to FIG. 10, a later screen shot 520 of the default settings screen is illustrated. At this stage, energy is being delivered from the console 102 to the stylus 104, as indicated by the “Energy ON” indicator light 509. The total treatment timer 512 of the graphical treatment progress display 514 shows that 12 seconds remain in the treatment. The outer ring 513 now includes a darker energy delivery time portion 511, and the lighter remaining portion of the ring 513 indicates the portion of the total treatment time that is still remaining. As the energy delivery stage of the treatment begins and progresses, the darker energy delivery time portion 511 takes up more and more of the outer ring 513, moving in a clockwise direction. In other words, the energy deliver indicator 511 starts at zero, at the twelve o'clock position on the ring 513, and moves around the ring in a clockwise direction.

Other indicators that the treatment is in progress include the temperature indicator 501 showing a temperature of 60 degrees Celsius and the treatment number indicator 504 showing that this is the first treatment being performed with the stylus 104 currently plugged into the console 102.

In some embodiments, the console 102 may be activated, and energy delivered to the stylus 104 in either of two ways—the start/stop button 510 may be touched, or a foot pedal coupled with the console 102 may be depressed. The “Energy ON” indicator 509 lights up when the console 102 is delivering power. The stylus type connected indicator 506 indicates what type of stylus 104 is connected to the console 102, which in the example shown is a Vivaer™ stylus (Aerin Medical, Inc., www.aerinmedical.com). This indicator 506 may be useful in embodiments where the console 102 is configured for use with multiple different types of styluses. The stylus temperature indicator 501 shows the actual temperature of the distal, treatment end of the stylus 104. The treatment number indicator 504 displays the number of the treatment currently being completed with the stylus 104 that is attached to the console 102. Finally, the custom treatment button 507 allows the user to customize one or more treatment parameters. Touching this button 507 will lead the user to a new display screen with different options. In alternative embodiments, the various icons and/or indicators on the default display 520 may be changed or moved. In some embodiments, one or more of the icons and/or indicators may be eliminated.

Referring now to FIG. 11, a third screen shot 530 of the default setting screen is illustrated. At a given amount of time into the procedure, the console 102 stops delivering RF energy to the stylus 104, and a cool down phase begins. The cool down phase is shown on the ring 513 as a differently colored or shaded segment 505, as compared to the Energy ON segment 511, and it may be called a cool down timer indicator 505. The cool down timer indicator 505 moves around the ring in a clockwise direction until the procedure is complete. Any amount of time remaining in the procedure is indicated by the total treatment time remaining portion of the ring 513.

In the screen shot of the main screen image 530 shown in FIG. 11, the current stage of treatment is illustrated as follows: The central circle total treatment timer 512 shows there are eight seconds remaining in the treatment. The Energy ON indicator segment 511 and the cool down timer segment 505 show that the energy delivery portion of the treatment is complete, because the cool down timer segment 505 has started. Since the total treatment time remaining portion of the ring 513 is still visible, this shows the user that the cool down phase is still in process. In a real life scenario, the cool down timer portion 505 would also be moving clockwise, thus easily telling the user what phase the treatment was in. In this case, the total treatment time portion of the ring 513 shows that approximately one fourth of the entire treatment time still remains. The temperature of 49 degrees Celsius in the temperature window also tells the user that the console 102 is in the cooling phase, since the temperature of the stylus 104 has decreased from 60 degrees. Finally, the Energy ON indicator light 509 is not illuminated.

In various embodiments, any colors, shades, shapes, graphics and/or the like may be used for the various segments 511, 505 of the outer ring 513. In one embodiment, for example, the Energy ON timer indicator 511 is navy blue, the cool down timer indicator 505 is gray, and the total treatment time remaining portion 513 is light blue. Any other colors may be used, however, in alternative embodiments. In another alternative embodiment, the entire ring 513 may be one color, and a line that acts as a timer may move clockwise around the ring 513, similar to a long hand on a clock. In a variation on such an embodiment, the color of the ring 513 behind the moving line may change. Thus, the ring 513 and the segments 511, 505 may have any suitable size, color scheme or configuration.

Additionally, the default (or custom) settings of the console 102 may have any suitable ranges and combinations for the various parameters of the console 102. For example, one timing default setting may have a total treatment time of 30 seconds, an Energy ON time of 18 seconds, and a cooling time of 12 seconds. This is but one example, however, and any number of other time settings may alternatively be used. A default temperature may also be set for energy delivery, for example 60 degrees Celsius as the maximum temperature. Again, any suitable default settings may be set in various embodiments.

Referring now to FIG. 12, as just described, the physician or other user can choose to access a custom treatment screen 630 by touching the custom treatment button 507 on the initial default screen 500 (FIG. 9). In the embodiment shown, the custom treatment screen 630 includes a graphical treatment progress display 600, which is smaller but otherwise the same as the graphical treatment progress display 514 of the default screen 500. The custom treatment screen 630 also includes a timer icon 601, an impedance display 602, a stylus icon 603, a number of treatments indicator 604, a cooling icon 605, a set cooling time window 606, a set Energy ON time window 607, a set temperature window 608, an actual temperature indicator 609, a down button 610, a back button 611, a stylus type indicator 612, an energy icon 613, an Energy ON indicator 614, a start/stop button 615, an up button 616, an actual power delivery indicator 617, a set power window 618, an energy power icon 619 and a temperature icon 620. As with the previously described default screen 500, the icons and/or indicators of the custom treatment screen 630 may be moved, changed and/or eliminated, according to various alternative embodiments.

Through the custom treatment screen 630, the user can adjust the power (power window 618), temperature (temperature window 608), treatment time (energy on time window 607) and/or cool down time (cooling time window 606), by touching any one of the set windows and then touching the up button 616 and/or the down button 610 to adjust a given value. To set power, for example, the user may touch the power window 618 and then adjust the temperature by pressing the up button 616 or the down button 610. The console 102 may be configured to only allow adjustments within ranges. For example, the power on the console 102 may be selected at 3 W, 4 W or 5 W in some embodiments. Maximum stylus temperature may be selected in a range of 50 degrees Celsius to 70 degrees Celsius in some embodiments. Energy delivery time (Energy ON time) may be selected for between 6 seconds and 18 seconds, in 2-second increments, and cooling time may be selected for between 0 seconds and 12, in 3-second increments, in one embodiment. Any other suitable ranges and combinations of ranges may be used, in alternative embodiments, and those provided here are merely examples.

For the information of the user, the impedance display 602 and stylus usage count 604 are also displayed. The back button 611 can be touched to return to the default screen 500 (FIG. 9). During treatment, the actual power 617 and temperature reading 609 are also shown. In alternative embodiments of the custom treatment display screen 630, one or more icons, indicators, buttons and/or windows may be moved, changed or eliminated.

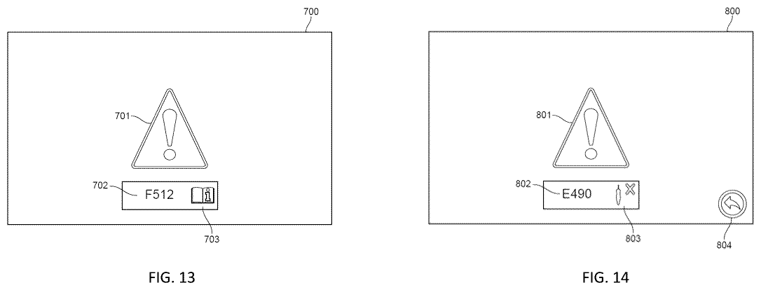

Referring now to FIG. 13, in some embodiments, the console 102 may be programmed to display a fault screen 700 when a fatal error of the system 100 occurs. The fault screen 700 may include, for example, a fault error symbol 701 that indicates a serious error has occurred, rendering the console 102 unusable. The fault screen may also include an error code 702, indicating what kind of error has occurred, and a refer to IFU (instructions for use) symbol 703. The fault error symbol 701 may be any color or combination of colors, such as a red triangle with a black exclamation point. In some embodiments, after the fault screen 700 appears, the console 102 may only be used after the user turns the console 102 off and turns it back on again.

Referring now to FIG. 14, in some embodiments, the console 102 may be programmed to display an error screen 800 when a non-fatal error of the system 100 occurs. The error screen 800 may include, for example, a caution symbol 801 that indicates an error has occurred. The error screen may also include an error code 802, indicating what kind of error has occurred, an error symbol 803, and a back button 804. The caution symbol 801 may be any color or combination of colors, such as a yellow triangle with a black exclamation point. In this embodiment, the user may touch the back button 804 to return to the previous screen being used when the error occurred, and the user may fix the error at that screen.

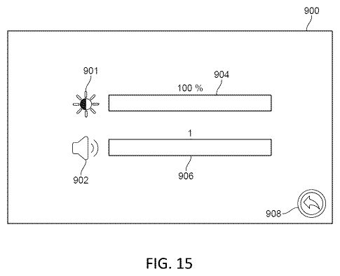

FIG. 15 is a screen shot of a settings screen 900, which may be provided on the display 108 of the console 102. In this embodiment, the settings screen 900 includes an adjust brightness icon 901, an adjust volume icon 902, a sliding bar brightness control 904 and a sliding bar volume control 906. The brightness control 904 and the volume control 906 may have any color or combination of colors. In some embodiments, a numerical indication of brightness and volume may also be included. The settings screen 900 also includes a back button 904 to allow the user to return to a previous screen.

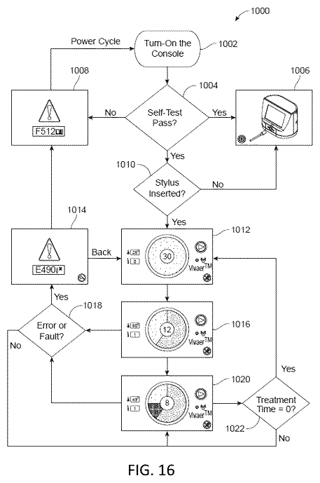

Referring now to FIG. 16, an exemplary method 1000 of using the console 102 of the electrosurgery system 100 is illustrated. First, the user turns on 1002 the console 102. The console 102 performs a self-test 1004. If the self-test passes, the console 102 displays the standby screen 1006. If the console 102 fails the self-test, the fault screen is displayed 1008. In some embodiments, the fault screen can only be cleared by power cycling the console 102 to repeat the self-test routine. Next, assuming the self-test is passed, the user plugs the stylus 104 into the console 102. If the correct stylus is inserted 1010, the console 102 shows the default main screen 1012. The user presses the start/stop button or a foot switch, and the RF energy delivery portion of the treatment starts 1016, followed by the cooling portion 1020. When the treatment is completed 1014, the screen returns to the default main screen 1012. If an error occurs during treatment 1018, the error screen is shown 1014. The user can press the back button on the error screen to return to the default main screen 1012. If the error is fatal, the screen changes to the fault screen 1008. At various points the in method 1000, the user may also select the custom treatment screen (FIG. 12) by pressing the custom treatment button 507 on the default screen.

### Treatment Device

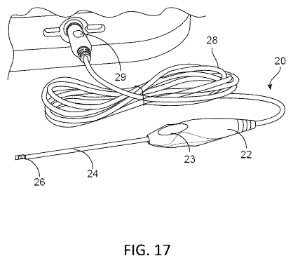

FIG. 17 is a more detailed view of the stylus 20. In this embodiment, the handle 22 includes an oval depression 23, which faces in the same direction as the treatment surface of the distal tip 26. The oval depression 23 allows the physician user to know the orientation of the treatment surface at all times and may also be used as a finger or thumb rest. In some embodiments, an on/off button may be positioned where oval depression 23 is illustrated on handle 22. This on/off button may be used to activate the stylus and thus provide energy to the electrodes at the distal tip 26. In some embodiments, the physician may use the handle on/off button as an alternative to a foot pedal for this purpose. The stylus shaft 24 may have any length suitable for reaching the posterior portion of the nasal cavity where the posterior nasal nerves reside. In one embodiment, for example, the shaft 24 is approximately 3.75 inches long, from its connection to the distal tip 26 at one end to its connection with the handle 22 at the opposite end. In some embodiments, the shaft 24 may have different lengths, for example between about 3 inches and about 4 inches in embodiments designed for addressing a posterior portion of the nasal cavity, and between about 1 inch and about 3 inches in embodiments designed for addressing an anterior portion of the nasal cavity, such as the nasal valve. The shaft 24 may also have any suitable width (or “diameter”). For example, in some embodiments, the shaft may have a width of about 4 mm to about 5 mm. The shaft 24 will be described in further detail below.

Referring now to FIGS. 18A and 18B, the distal tip 26 is shown in detail. The distal tip 26 includes a treatment surface 30 (or “tip face”), two rows of four bipolar RF electrodes 32, and a thermocouple 34 between the electrodes 32. As illustrated in FIG. 18A, the bottom surface 36 of the distal tip 26 may be tapered or slanted, to give the distal end of the distal tip 26 a narrower profile to facilitate insertion and advancement of the distal tip 26 into the nostril and to the posterior portion of the nasal cavity. The treatment surface 30 is made of a non-conductive material (e.g., plastic) and may have a slightly convex shape, as illustrated, or may alternatively be flat, concave, or more convex than the shown in FIG. 18B. The slightly convex shape may help ensure contact of the electrodes 32 and the thermocouple 34 with mucosal tissue during use.

In some embodiments, the thermocouple 34 may be removed or replaced by a different type (or shape or number) of temperature sensor(s). The system can use one or more temperature sensors to monitor temperature of the treated tissue or adjacent tissues to monitor for changes or to execute a tissue apposition check or monitor other biometric information. The thermocouple 34 (or any temperature sensor) is used to measure temperature of the mucosa and provide the measurements to a processor in the console 12, which can in turn automatically adjust whether and how much energy is delivered to the stylus 20. In some embodiments, the distal tip 26 or a portion thereof may be translucent, to facilitate visualization of target tissue and/or nasal mucosa.

The system can also monitor the impedance of the tissues in contact with the device to monitor for changes or to execute a tissue apposition check or monitor other biometric information. The system may include an optical generator that enables an optical signal, possibly in the near infrared spectrum, to be sent to the tissue and enables monitoring of the treated tissues by confirming tissue has been altered through some property shift, for example, a decrease in oxygenation may indicate that irreversible electroporation has occurred in the targeted tissue.

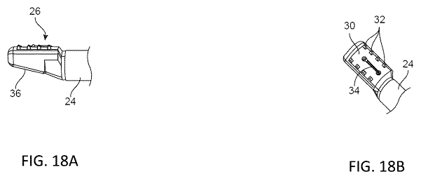

FIGS. 19A and 19B illustrate, in a side view and a front view, respectively, a distal portion of an alternative embodiment of a nasal airway tissue treatment stylus 130. In this embodiment, the height or thickness of the distal tip 134 is thinner than in the previously described embodiment and thinner than the shaft 132 of the stylus 130. The distal tip 134 includes a treatment surface 136, four pairs of bipolar RF electrodes 138, and a thermocouple 139. The thinner distal tip 134 may facilitate passage of the tip 134 through a nostril and around nasal cavity anatomy for positioning at a treatment site in a posterior portion of the nasal cavity. In all other respects, the distal tip 134 may include any of the features of the embodiment described above.

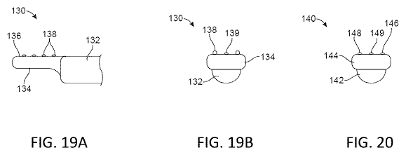

FIG. 20 is a front view of yet another embodiment of a nasal airway tissue treatment device 140. This embodiment also includes a shaft 142 and a distal tip 144, which in turn includes a treatment surface 146, four pairs of bipolar RF electrodes 148 and a thermocouple 149. In this embodiment, however, the electrodes and thermocouple are flat, rather that rounded or pointed. They protrude only slightly from the treatment surface 146. This further helps reduce the overall height or thickness of the distal tip 144 and thus makes it easier to advance the tip 144 through the nostril and nasal cavity. In some embodiments, the electrodes and thermocouple may even be completely flush with the treatment surface.

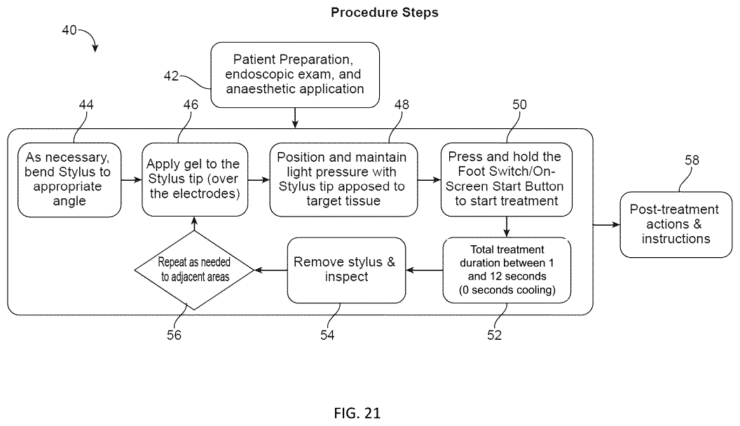

FIG. 21 illustrates an exemplary embodiment of a method 40 for using the treatment system 10 to treat nerve tissue (and/or other tissues) in the nasal cavity to treat rhinitis. In this embodiment, the method 40 includes the steps of: preparing the patient 42, such as with local anesthesia and an endoscopic examination of the nasal cavity; bending the stylus 44, if desired (explained further below); applying conductive gel to the stylus distal tip 46, over the electrodes; advancing the distal tip of the stylus into the nostril and contacting it with the treatment area 48, then maintaining light pressure with the distal tip against the treatment tissue; pressing and holding down the foot switch 50 to start the treatment; continuing to hold down the foot switch for twelve seconds 52 (the treatment time in this embodiment); removing and inspecting the distal tip 54; and repeating 56 steps 46, 48, 50 and 52 as necessary, to treat additional treatment areas. In one embodiment, for example, it is recommended that at least three, twelve-second treatments are performed along the course of the posterior nasal nerve. Any other number of treatments may be performed, however, in alternative embodiments. In some embodiments, each treatment may last a different period of time than 12 seconds, such as between 1 second and 120 seconds, or between 10 seconds and 60 seconds in various embodiments. For example, PFA treatments can be between 1-2 seconds of treatment. For example, PRF treatments can be up to 120 seconds. When a treatment on a patient is complete, a final step may be to provide post-treatment actions and instructions to the patient 58.

The method may be repeated for as many treatment areas as desired. In some embodiments, the stylus 20, the console 12 or both may be configured to allow only a certain number of treatments for any given stylus 20. This may help prevent reuse of the stylus 20 on multiple patients or overtreatment of any one patient. For example, in one embodiment, the stylus 20 may only be able to deliver sixteen 12-second treatments. In other embodiments, the stylus 20 may be capable of delivering ten to thirty 12-second treatments, for example. In yet other embodiments, the stylus 20 may be capable of delivering any number of treatments, but the console 12 is able to identify each stylus 20 and count or identify how many treatments have been applied with that stylus 20. The console 12 may be configured to shut down or simply not deliver energy to a stylus 20 that has reached its maximum number of allowed treatments. In other embodiments, a single stylus 20 may be used with the console 12 to deliver as many treatments on one patient as desired, but once the treatment on that patient is completed, the stylus 20 is rendered inoperable for use with any additional patient(s). Inoperability may be conferred by a computer chip in the handle 22 of the stylus 20, or alternatively the console 12 may destroy or alter a portion of the stylus 20 when the stylus 20 is unplugged from the console 12 or at some other point at the end of a treatment.

In some embodiments, the nasal airway tissue treatment stylus 20 may be used for treating several different types of target tissue in one patient. Before treating posterior nasal nerve tissue, after treating that tissue, or both, one or more additional tissues may be treated. Such tissues include mucosa, nerves and/or other tissue of any one of the nasal turbinates, nasal swell bodies, the nasal septum, and mucus producing cells anywhere in the nasal cavity. Therefore, the treatment method illustrated in FIG. 21 may be added to by treating any one or more of these additional tissues. Physicians may often treat the inferior and/or middle turbinate before or after proceeding with the steps in FIG. 21 to treat posterior nasal nerve tissue.

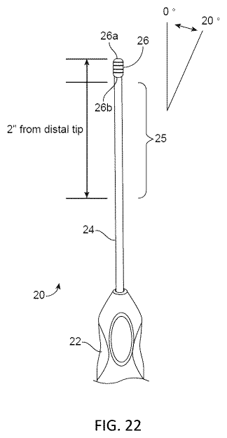

Referring next to FIG. 22, a portion of the stylus 20 is shown, illustrating features of the shaft 24. In this embodiment, the shaft 24 is malleable, so that it can be bent by a physician user and retain the bent shape during use. This may be especially advantageous, for example, in helping the physician advance the distal tip 26 of the device around anatomical structures to a posterior target area in the nasal cavity. As illustrated in FIG. 22, the distal tip 26 has a distal edge 26a and a proximal edge 26b. Just proximal to the proximal edge 26b of the distal tip 26 is a bend section 25 of the shaft 24. The bend section 25 is approximately 2 inches in length in this embodiment, and it is the section of the shaft that is recommended for bending. Physician users may be instructed, for example with instructions for use provided with the system 10, to bend the shaft 24 only within the bend section 25 and not too close to the distal tip 26. This may help prevent weakness at the areas where the shaft 24 is connected to the distal tip 26 and the handle 22. In the embodiment shown, the entire shaft 24 is made of a malleable material, but in alternative embodiments only a portion of the shaft 24 might be malleable, such as the bend section 25.

The shaft 24 may be manually bent by the physician to the appropriate bend angle. While bending, the physician should support the stylus 20 by the shaft 24, not by the handle 22 or the distal tip 26. The bend should be formed in the orientation the electrodes 32 are facing. The shaft 24 may be bent to any suitable angle. In one embodiment, however, it is recommended that the shaft 24 only be bent to a maximum of approximately 20 degrees away from the longitudinal axis of the stylus 20. Again, this limit on bending may help maintain the structural integrity of the stylus 20. Bending the shaft 24 at all is entirely optional, and some or even all physicians might decide not to bend the shaft 24 at all. In alternative embodiments, the shaft 24 might be rigid and not malleable. In general, all parts of the stylus 20, other than the electrodes 32 and the thermocouple 34, may be made of non-conductive materials, such as any suitable plastic or polymer.

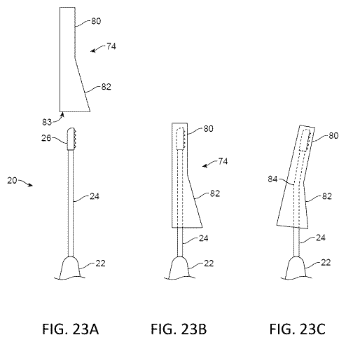

Referring to FIGS. 23A, 23B, and 23C, in some embodiments a bending tool 74 may be provided for bending the shaft 24 of the stylus 20. In this embodiment, the bending tool 74 includes a narrow distal portion 80, a wide proximal portion 82, and an opening 85 into which the distal tip 26 and a portion of the shaft 24 enter. As illustrated in FIG. 23B, the bending tool 74 may first be slid down onto the stylus 20. The bending tool 74 may then be tilted, as shown in FIG. 23C, to form a bend 84 in the shaft 24. The bending tool 74 may have any suitable length, to bend the shaft 24 in a desired location. For example, in some embodiments the bending tool 74 is designed to bend the shaft 24 somewhere between one third and one half of the length of the shaft measured from the handle 22. Alternative embodiments of the bending tool 74 may have any other suitable size, shape and configuration for bending a shaft 24 of a stylus 20.

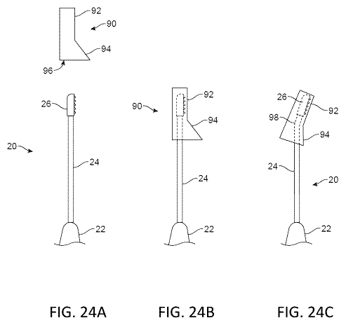

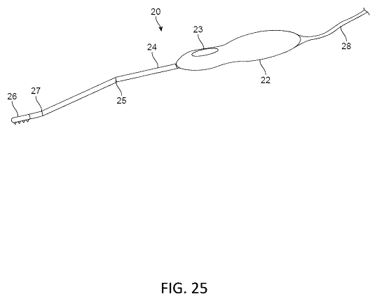

FIGS. 24A, 24B, 24C illustrate the use of another embodiment of a shaft bending tool 90. In this embodiment, the shaft bending tool 90 includes a narrow distal end 92, a wide proximal end 94, and an opening 96, as with the previous embodiment. In this embodiment, however, the shaft bending tool 90 is significantly shorter than the previously illustrated embodiment. As illustrated in FIG. 24C, this shorter bending tool 90 creates a bend 98 in the shaft 24 that is much closer to the distal tip 26. Physicians may want to bend the shaft 24 of the stylus 20 in different locations. Thus, in some embodiments more than one size or shape of bending tool 74, 90 may be provided. A physician may select one bending tool 74, 90 to make one bend, or she may use two (or more) different bending tools 74, 90 to make multiple bends in the shaft 24.

Referring to FIG. 25, as just mentioned, in some cases a physician may bend the shaft 24 of the stylus 20 at two different locations, forming for example a proximal bend and a distal bend. In some embodiments, the shaft 24 may include one or more proximal bend markers 25 and one or more distal bend markers 27. Bend markers 25, 27 may be located at any location along the length of the shaft 24 and may be made with paint, etched into the surface, or by any other means. In the illustrated embodiment, two bends are made to give the shaft 24 a bayonet shape, as desired by some physicians to navigate around anatomical structures in the nasal cavity while still positioning the treatment surface of the distal tip 26 in full contact with nasal mucosa on the lateral wall of the nasal cavity. It bears repeating that in various alternative embodiments, the shaft 24 may include any number of bend markers 25, 27 at any locations and/or the shaft 24 may be bent (with or without markers) at any suitable location or at multiple locations.

In various embodiments, the shaft 24 may be either more or less malleable, depending on the desired stiffness versus bendability of the shaft 24. In some embodiments, only certain portions of the shaft, which are designed to be bent, are malleable, while others are stiff. Or certain portions may be more malleable than others. More malleable sections may have a thinner wall than less malleable sections and/or the shaft 24 may be made of different materials in different sections. The latter is likely to complicate manufacturing and increase expense, however, so in at least some embodiments the shaft 24 is made of one piece of material, such as a metal hypotube. In such cases, differences in malleability may be achieved via differences in wall thickness, material selection, material processing, or material heat treatment.

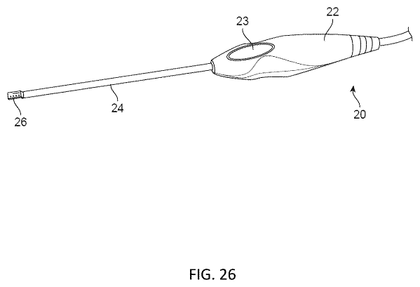

FIG. 26 is another illustration of the stylus 20, showing how the oval depression 23 on the top of the handle 22 aligns with the treatment surface 30 of the distal tip 26. This allows the physician to always know what direction the treatment surface 30 is facing. The oval depression 23 is configured to provide an ergonomic location for placement of the physician's thumb, but depressions of different shapes may be used in different embodiments. Other alternative embodiments may use any other suitable directional indicator on the handle 22 and/or a proximal portion of the shaft 24, to show direction of the distal tip 26.

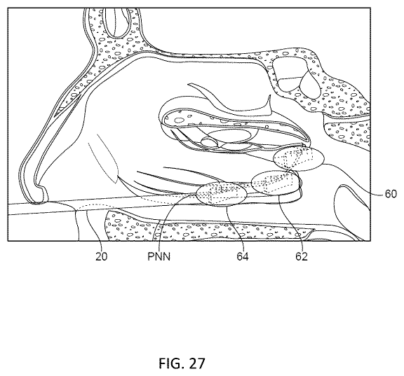

FIG. 27 is a sagittal view of the nasal cavity, showing three treatment areas for treating the nasal cavity with the stylus 20. In this embodiment, the stylus 20 has been used to deliver energy to a first target location 60, a second target location 62 and a third target location 64, all of which fall along the path of the posterior nasal nerve PNN. Additional treatments may optionally be provided in the same general area and/or in different areas of the nasal cavity. For example, some physicians may use the stylus 20 to treat a nasal septum, a septal swell body, an inferior turbinate and/or other soft tissue(s) in the nose, all as part of the same treatment on one patient. FIG. 27 is thus provided for illustrative purposes only.

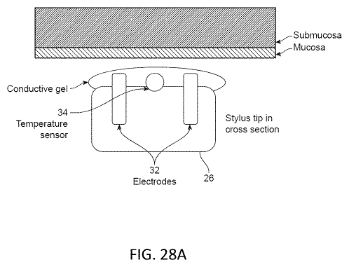

Referring to FIGS. 28A and 28B, two steps of the method for using the nasal tissue treatment system 10 are illustrated diagrammatically. In FIG. 28A, the distal tip 26 of the stylus 20 is shown in an end-on, cross-sectional view, showing two electrodes 32 and the temperature sensor 34. The distal tip 26 has conductive gel on its treatment surface 30 and is in position within the nostril, near the mucosa. Next, as illustrated in FIG. 28B, the physician contacts the mucosa with the distal tip 26, applies gentle pressure against the mucosa, and presses the foot pedal to activate the stylus 20 and deliver energy from one set of electrodes 32, through the mucosa, to target tissue(s) in the submucosa, and back to a second set of electrodes 32. This process generates heat and treats the target submucosal tissues, such as the posterior nasal nerve and/or other nerves. Again, the treatment may be timed by the console 12, which may automatically stop the delivery of energy after a preset time, such as 12 seconds in one embodiment. The thermocouple 34 measures the temperature of the mucosa it is contacting, and the system 10 uses this measurement to automatically shut off the system 10 if a temperature is too high or otherwise outside of a preset range of acceptable temperatures.

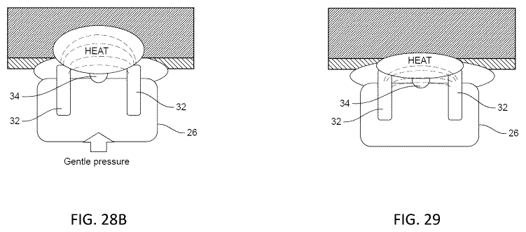

FIG. 29 illustrates the distal tip 26 in a similar position as shown in FIG. 28B, but in this case the physician is not applying gentle pressure to the mucosa with the distal tip 26. As illustrated, it is possible that without application of pressure, the applied energy will not penetrate the submucosa to reach the desired target tissue. In some embodiments, the stylus 20 is designed to determine whether adequate pressure is being applied and to only activate the electrodes 32 when the pressure is applied.

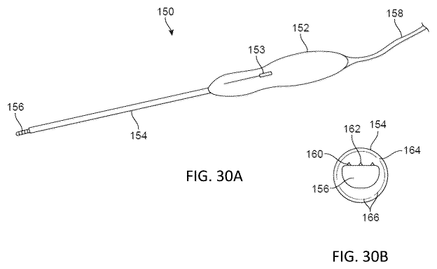

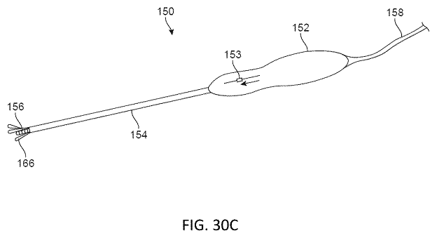

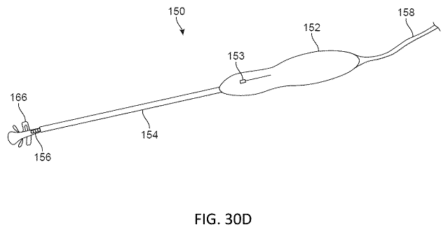

Referring now to FIGS. 30A-30D, another embodiment of a nasal airway tissue treatment stylus 150 is illustrated. As shown in FIG. 30A, the stylus 150 includes a handle 152 with a slider 153, a shaft 154, a distal tip 156 and a power cord 158. FIG. 30B is a front view of the stylus 150, showing additional features. In this embodiment, the stylus 150 includes a space 164 between the inner wall of the shaft 154 and the outer perimeter of the distal tip 156. (FIG. 30B also shows the two rows of electrodes 160 and the thermocouple 162 of the distal tip 156.) Within the space 164 and around the distal tip 156 resides an expandable wire electrode component 166, which is moveable out of the distal end of the shaft 154 to allow it to expand and be used for delivering a treatment.

In use, the stylus 150 may be used first in the configuration shown in FIG. 30A. The electrodes 160 of distal tip 156 may be used to treat tissue, such as posterior nasal nerve tissue, turbinates, nasal swell bodies and/or the like. In one embodiment, for example, the distal tip 156 may be used to treat one or more nasal turbinates in areas that are anterior to the nasal nerve that will be treated subsequently.

After treating with the stylus 150 as shown in FIG. 30A, and referring now to FIG. 30B, the slider 153 on the handle 152 may be advanced to advance the expandable wire electrode component 166 out of the distal end of the shaft 154. FIG. 30D shows the slider 153 further advanced and the expandable wire electrode component 166 fully advanced and expanded. The expandable wire electrode component 166 may include multiple bipolar electrode pairs located at various locations along its length. Optionally, the expandable wire electrode component 166 may also include one or more thermocouples and/or multiple nerve sensors (such as electrodes), which may be used to measure a temperature of nasal mucosa and sense where nerve tissue is located under the mucosa, respectively. In some cases, all electrode pairs may be activated at the same time. Alternatively, only one or more selected electrode pairs may be activated. In some cases, the expandable wire electrode component 166 may be used to determine the location of nerves and then only bipolar electrode pairs located directly over those nerves may be activated.

In one example, after the stylus 150 has been used in the initial configuration shown in FIG. 30A to treat one or more turbinates, swell bodies or the like, the stylus 150 may be changed to the expanded configuration shown in FIG. 30D. In that configuration, the expandable wire electrode component 166 may be used to treat posterior nasal nerves and/or any other nerve tissue the physician wants to treat. In other words, the stylus 150 is used to treat nerve tissue in the FIG. 30D configuration and used to treat any other tissues in the FIG. 30A configuration. This is only one example, however, and a physician may use the stylus 150 in any suitable manner. After treatment with the expandable wire electrode component 166 is complete, the expandable wire electrode component 166 may be pulled back into the shaft by sliding the slider 153 proximally. It may also be possible to treat further in the FIG. 30A configuration after treatment with, and retraction of, the expandable wire electrode component 166.

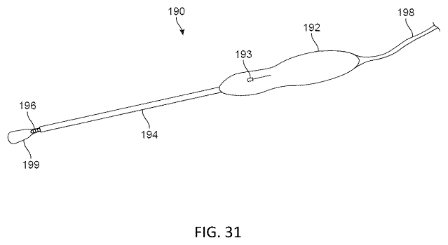

Referring now to FIG. 31, an alternative embodiment of a nasal airway treatment stylus 190 is similar to the one just described, except that rather than the expandable wire electrode component 166, this embodiment of the stylus 190 includes an expandable cryotherapy balloon 199. The stylus 190 also includes a handle 192 with a slider 193, a shaft 194, a distal tip 196 and a power cord 198. In this embodiment, the cryotherapy balloon 199 can be advanced out of the distal end of the shaft 194 using the slider 193. A source of cryogenic substance, such as nitrous oxide in a small canister, may be attached to the handle 192 and transmitted through a lumen in the shaft 194 to inflate the cryotherapy balloon 199. The inflated cryotherapy balloon 199 will absorb energy from the nasal airway tissues and thus can be used to ablate posterior nasal nerves and/or any other target nerve tissue or other target tissues. When a treatment is complete, the cryogenic substance may be evacuated from the cryotherapy balloon 199, causing it to deflate, and the stylus 190 may be removed from the nose. Optionally, the deflated cryotherapy balloon 199 may be pulled back into the shaft 194 before removal of the stylus 190, but that may not be necessary. As with the previous embodiment, any suitable nasal airway tissue may be treated with the distal tip 196 of the stylus 190 before inflating the cryotherapy balloon 199 for additional tissue treatment.

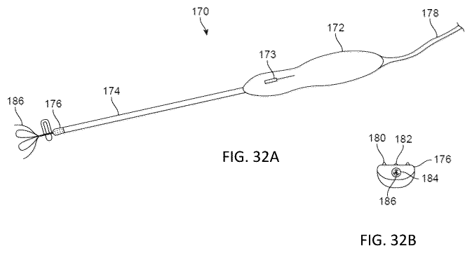

With reference now to FIGS. 32A and 32B, another alternative embodiment of a nasal airway tissue treatment device 170 is illustrated in perspective view (FIG. 32A) and front view (FIG. 32B). In this embodiment, the stylus 170 includes a handle 172 with a slider 173, a shaft 174, a distal tip 176, an expandable wire electrode component 186, and a power cord 178. As best seen in FIG. 32B, the distal tip 176 includes two rows of bipolar RF electrode pairs 180, a thermocouple 182 and an opening 184 which leads directly into a lumen in the shaft 174. The expandable wire electrode component 186 is located inside the opening 184 and the lumen (not visible on these drawings) and may be advanced out of the opening 184 with slider 173 to allow the expandable wire electrode component 186 to expand and be used for treatment. All of the same features and methods of use described above may be applied to this embodiment of the stylus 170, the primary difference being that the expandable wire electrode component 186 is located within the distal tip 176 and advances out of the opening 184. The expandable wire electrode component 186 may have any suitable size, shape and configuration of wires, according to various embodiments.

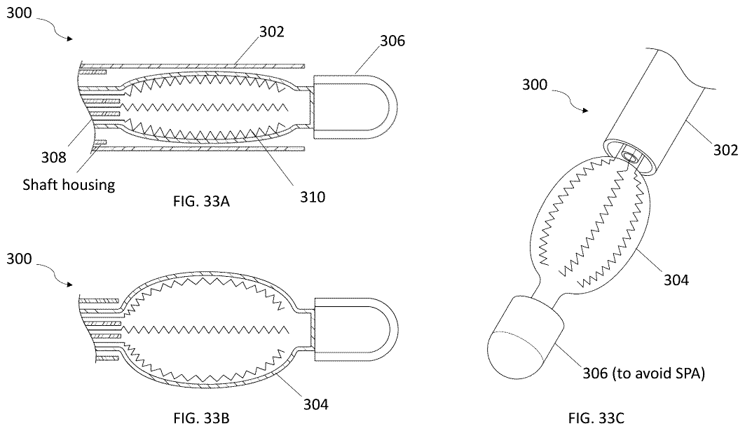

FIGS. 33A-33C illustrate exemplary embodiments of a treatment device having an expandable wire electrode component with a plurality of electrodes thereon for delivering energy to the target nasal tissue. In some embodiments, the treatment device 300 in is in the form of a sheath balloon. The treatment device 300 includes an elongate sheath 302 with an opening at a distal end such that an expandable member 304, such as a balloon, can extend therefrom. In some embodiments, the balloon is configured to expand asymmetrically. A standoff 306 is coupled to a distal end of the balloon, which can in the form of a soft-tipped component or in the form of a wire loop. An inflation lumen 308 extends thought the shaft and couples to a proximal end of the balloon is allow the balloon to move from an uninflated configuration (FIG. 33A) to an inflated configuration (FIGS. 33B and 33C) to allow one or more electrodes 310 positioned on the outer surface of the balloon to contact a target tissue for treatment. The one or more electrodes 310 can be positive or negative, and can be symmetrical or asymmetrical. The shape of the electrodes can vary, but the electrodes are configured to expand as the balloon is inflated.

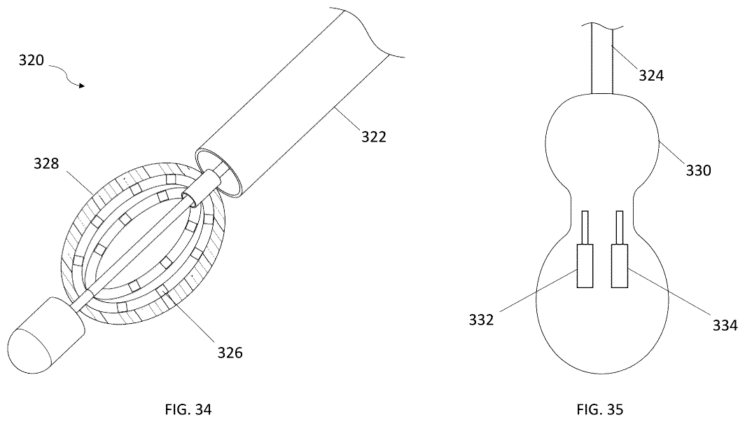

FIGS. 34 and 35 illustrate exemplary embodiments of a treatment device having a multi-electrode treatment element with multiple channels can be implemented in a symmetrical or asymmetrical basket. The electrode component includes a plurality of sections that are all coupled together at a distal end and a proximal end. In some embodiments, the wire electrode component is configured to deploy into the nasal cavity and expand and conform to the target treatment tissues. In some embodiments, the treatment device 320 in is in the form of a partial and/or asymmetrical basket. The treatment device 320 includes an elongate sheath 322 with an opening at a distal end such that an expandable basket 324 can extend from a distal end of the sheath. The basket can be deployable (either delivered from a sheath or delivered in a collapsed state) and extend from the distal opening of the sheath. Each segment of the basket can include a plurality of electrodes 326 thereon. The basket can also include an optional septal wall shield 328 that can be deployable to protect the basket and the electrodes during use. A handle 330 can be coupled to the proximal end of the shaft and includes features thereon for controlling the basket. In some embodiments, the handle includes an activation device that can function as a slider 332 to control the movement of the sheath relative to the basket. In some embodiments, the handle includes an activation device that can function as a slider 334 to control deployment of the basket/electrodes.

In some embodiments, the asymmetric basket can be seated against the turbinates and the septal wall. A balloon can be used to deploy the electrodes to deliver energy to a target nasal tissue. In some embodiments, the device allows for conformability with multiple electrode leaflets distributed across the device. Further, the tip length can be “inactive” to ensure that distal portion acts as a standoff from tissues not intended for treatment (for example, the distal tip can be 3 mm to 5 mm).

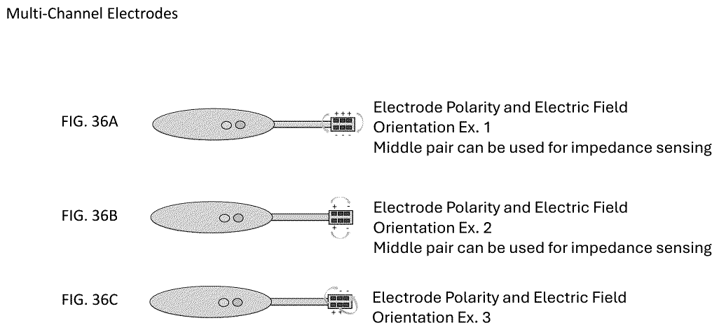

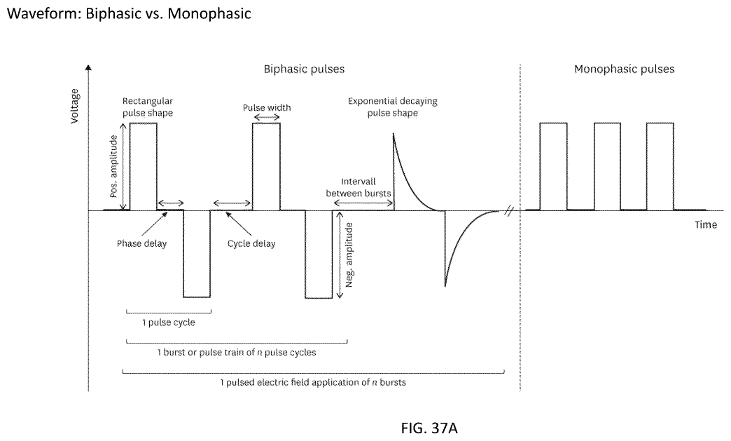

FIGS. 36A, 36B, and 36C illustrate exemplary embodiments of multi-channel electrodes. FIG. 36A illustrates an orientation of electrode polarity and electric field having a middle pair of electrodes used for impedance sensing. In some embodiments, the device can sense impedance which may enable tissue detection and provide treatment alterations to avoid directly delivering energy to a non-target tissue. In some embodiments, the treatment algorithm can monitor the impedance and deliver energy until a certain impedance is reached or a bioelectrical signal is no longer sensed. FIG. 36B illustrates another orientation of electrode polarity and electric field having a middle pair of electrodes used for impedance sensing. FIG. 36C illustrates another orientation of electrode polarity and electric field.

### Energy Types

As explained above, various types of energy can be used with the systems and devices described herein to treat nasal tissue to treat rhinitis, nasal obstructions, migraines, other headaches, and other nasal diseases, but in some embodiments, pulsed electric field energy can be used for conducing pulsed field ablation (or “PEF ablation”) or using pulsed radiofrequency energy (PRF energy) of one or more tissues, such as but not limited to nerve tissue and various nasal tissue and structures. For example, the energy generator can be in the form of a PEF generator and the various embodiments of electrodes may be operated as PEF ablation electrodes. In any given embodiment, the pulsed electric field generator may be configured to provide exponentially decaying, square wave, bipolar, monophasic, biphasic, and/or oscillatory pulses.

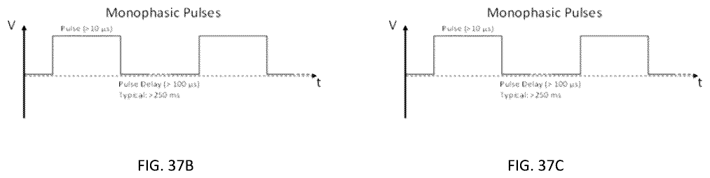

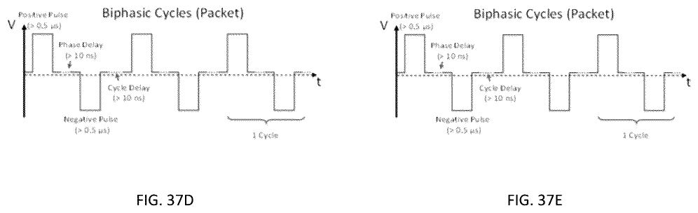

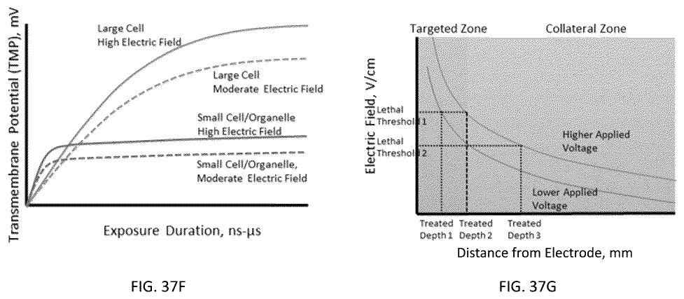

In some embodiments, the radiofrequency pulses can be delivered using high frequency alternating pulses. In some embodiments, the electrosurgical generator may be configured to output waveforms with high-voltage pulses of alternating direct current with pulses delivered as a pulse train. In some embodiments, the pulses can be monophasic or biphasic. Monophasic pulses are single polarity pulses, where biphasic pulses consist of positive and a negative pulse amplitude. FIGS. 37A-37G illustrate exemplary graphs showing biphasic and monophasic pulses.

In some embodiments, a pulse train includes one or more monophasic or biphasic pulses delivered as a specified number of pulses with a prescribed pulse width, pulse amplitude, pulse cycle with a pulse delay and a cycle delay. The pulse delay, or pulse interval, is the duration following each pulse delivery of specified pulse width and pulse amplitude. The pulse cycle is comprised of both a positive and a negative pulse.

In some embodiments, the pulse train may be delivered in repeated intervals to create a burst, which is a string of pulse trains with some interval between pulse trains to allow for tissue to recover and to minimize thermal effects. The high-voltage pulses may be delivered using rapidly alternating voltages that are positive and negative (biphasic) or positive only (monophasic). The use of biphasic pulses has been shown to reduce the possibility of muscle stimulation when compared to monophasic pulses.

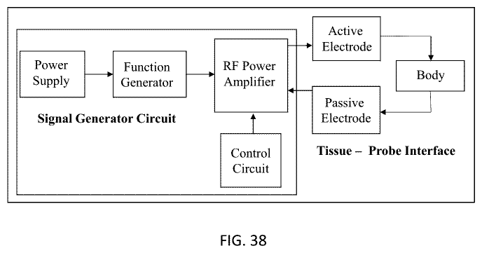

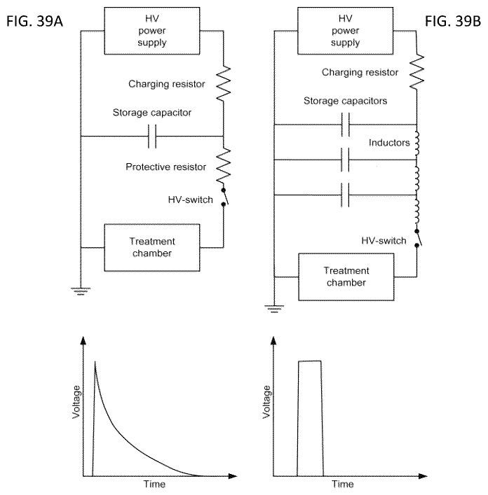

FIGS. 38, 39A, 39B, and 40 illustrate various embodiments of system diagrams and circuits for producing the pulsed energy delivered to the various treatment devices described herein. FIG. 38 illustrates an exemplary embodiment of a RF generator system that could have some waveform generated by the function generator. FIGS. 39A and 39B illustrates exemplary embodiments of circuits used in Pulsed Electric Fields generators to generate a square wave or a decaying wave.

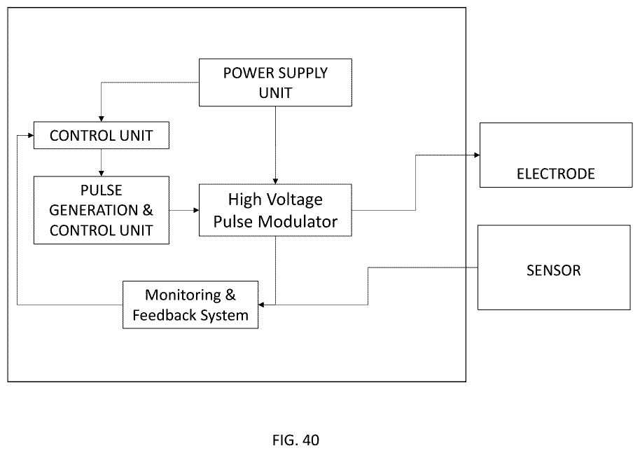

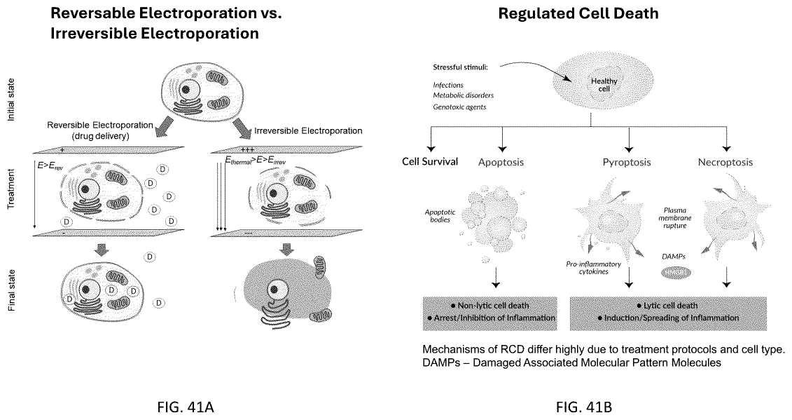

FIG. 40 illustrates an exemplary embodiment of a charging and switching circuit that could be employed, and that includes a sensor to allow for monitoring temperature to ensure that targeted tissue or some proxy measurement is not exceeded. In some embodiments, the power supply unit is configured to provide power voltages to the high voltage pulse modulator. In some embodiments, the power supply unit generates and provides power voltages which have a voltage level that is controlled by a control signal from the control unit. The control unit can be configured to generate and provide one or more control signals to pulse generation and control unit and to power supply based at least partly information from the monitoring and feedback system regarding temperature. in some embodiments, the control unit, power supply 1560, pulse generation and control unit, the high voltage pulse modulator, and the monitoring and feedback system can form a feedback loop which causes one or more characteristics of the pulse to be changed based on the feedback information from various sensors.

### Pulsed RF

In some embodiments, the generator may be configured to deliver radiofrequency energy pulses such that there is minimal thermal effect on the targeted tissues and a plurality of pulses are delivered for a specified time while the surface temperature is monitored using a sensor such as a thermocouple to ensure there is minimal thermal effect. As explained above, pulsed radiofrequency energy is typically used for neuromodulation.

It was found that pulsed radiofrequency impacts many different biological pathways involved in the modulation of chronic neuropathic pain (neuralgia). With regards to nociceptive signaling, PRF treatment modulates ion channels (Na/K ATPase, HCN, P2X3), CGRP, neurotransmitters (aspartate, citrulline, M-ENK, glutamate), postsynaptic receptors (AMPA-R, GABA-B), and synaptic function (KCC2). PRF treatment also modulates immune activity, including microglial markers (CD3, CD56, Iba1), inflammatory cytokines (IL-6, IL-17, IRF8, IFN-γ, TNFα), and intracellular proteins implicated in immune mediated neuropathic pain (BDNF, β-catenin, JNK, p38, ERK1/2). For example, the proposed mechanism of action of PRF pertaining to pain relief relies on a decrease in pro-inflammatory cytokines, an increase in cytosolic calcium concentration, a general effect on the immune system, and a reduction in the formation of free radical molecules.

For example, the alternating pulses can be in the range of 300-500 kHz and pulsed in the range of 1-50 milliseconds at a pulse frequency of 2-5 Hz with voltage amplitudes ranging from 0-100V. The pulses can be delivered for a duration of 10 to 300 seconds.

The system can also employ a temperature control setpoint that is measured during energy delivery. The energy delivery ceases until the tissue temperature drops below a specified amount below the temperature control setpoint. Once reached, the pulsing continues to be delivered through the duration of treatment. In some embodiments, the temperature control setpoint or pulse parameters may be adjusted to reach a desired effect on the tissue. In some embodiment, the temperature setpoint can be above 40 degrees Celsius. In some embodiments, to produce a more durable effect with longer lasting effect, the temperature setpoint can be between 50 degrees Celsius to 70 degrees Celsius. In some embodiments, the temperature setpoint can be between 50 degrees Celsius to 55 degrees Celsius. In some embodiments, the temperature setpoint is up to 50 degrees Celsius or up to 55 degrees Celsius. One exemplary setting has the energy being delivered using 20 ms pulsed at 45V at a pulse frequency of 2 Hz for less than 120 seconds with a temperature control setpoint at 42° C.

For example, pulse trains for PRF are typically around 500 khZ (i.e., 10K cycles/burst at 20 ms). The pulse rate is 2-5 hz (2-5 pulses/second). The pulse width is typically 20 ms. The treatment duration is 120-360 sec. The voltage is between 45-50V. The temperature control point is 42° C. Variation on pulse parameters can be adjusted to potentially alter the therapeutic effect, for example, treating the posterior nasal nerve with a higher voltage and increasing temperature control point may produce sufficiently durable effects. Nerves have been shown to be impacted at temperatures below 60 degrees C. It is possible for a pulse train to be delivered that will produce a durable effect that may last longer than typical PRF chronic pain treatments (e.g. greater than 1 year).

### PFA

In some embodiments, the generator may be configured to deliver PFA energy to the tissue. PFA utilizes high voltage pulsing that induces regulated cell death through irreversible electroporation of the targeted cells. FIGS. 41A and 41B illustrate the mechanism of electroporation and cell death that can be achieved using PFA.

PFA high-voltage pulses can be used to induce irreversible electroporation that creates pore formation in the cellular membrane. This causes biochemical signals to trigger cell death via apoptosis (non-necrotic) or other means of regulated cell death. The large electric fields from PFA energy cause destruction of the nerve function through axonal disintegration and demyelination. There may be some localized effect of electrodes heating the tissue through resistive heating that creates some necrotic lesions, but the primary pathway of the cell death is through apoptosis.

For example, pulse trains for PFA can have a voltage field of 3800 V/cm (rat sciatic nerve); also pig study treated with 1500 V/cm. The pulse type can be biphasic or monophasic square waves. The pulse width is 100 microseconds (square). The pulse rate is 10-50 kHz (note: the pulse delay and cycle delay could be varied to allow any thermal energy to dissipate). The pulse count is 10-90+ (10 pulses for rat sciatic, 90 pulses for pig). The applications can be for nerves. Variations on pulse parameters could have an impact on the treatment effect.

For example, PFA typically uses voltages 1-3 kV, pulse widths from nano to milliseconds. The number of pulses can be dependent on type of tissue being treated.

For example, the voltage amplitude may range from 1 kV-10 kV such that the electric field strength can range from 0.5 kV/cm to 10 kV/cm with pulse widths ranging from 0.05 microseconds to 200 microseconds with the number of pulses varying from 4 to 200 pulses per pulse train.

The systems and devices described herein can employ PFA as a non-thermal method to target and ablate nerve tissues. The pulsed field ablation techniques can be used to induce reversible or irreversible electroporation which can lead to Regulated Cell Death of the targeted tissue. The system would promote an immune response that could lead to a reduction in inflammation in the treated areas. The pulse waveforms delivered can be monophasic, biphasic, monopolar, bipolar and asymmetric. The pulse duration can be on the order of nanoseconds to milliseconds delivered with a plurality of voltages for the pulse amplitudes and delivered in a pulse train (bursts of packets of pulses). The electric field generated can range from 20 V/cm up to 100 kV/cm. The pulse train can be formulated to prevent muscle stimulation/contractions while still delivering a sufficient amount of energy to induce irreversible electroporation.

For PFA, the temperature control setpoint is less important since the energy is delivered so quickly and minimal resistive (joule) heating occurs. In some embodiments, the temperature can be monitored during delivery of PFA energy, which can reduce some of the risk for any undesired thermal buildup attributed to resistive. In some embodiments, thermal buildup ca also be mitigated by increasing the pulse delay as a longer cycle time allows for more tissue recovery time between pulses.

### Methods of Treatment

This application describes various aspects and embodiments of a device, system, and method for treating tissue, including nerve tissue.

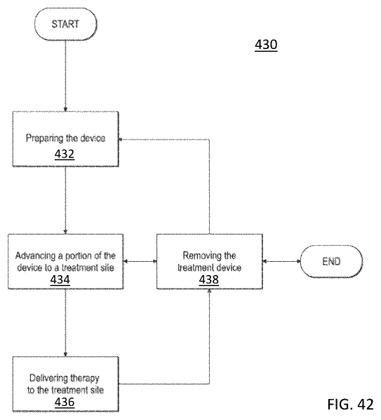

FIG. 42 is a flow diagram of an example method 430 of use of the various devices disclosed herein. In certain implementations, the method 430 may include preparing the device in step 432, advancing a portion of the device to a treatment site in the nasal cavity in step 434, delivering therapy to the treatment site in step 436, and removing the treatment device in step 438.

In some embodiments, step 432 of preparing the device may include removing the device from sterile packaging, assembling one or more components of the device, sterilizing the device, attaching the device to an energy source, and/or other preparatory work. In certain implementations, this step may include customizing the device to suit the particular needs of the patient and the clinician. This may include articulating, manipulating, or otherwise changing one or more components of the device.

The step 434 of advancing a portion of the device to a treatment site in the nasal cavity may follow the preparation of the device in step 432. For example, the clinician may navigate the patient's nasal anatomy to advance a treatment portion of the treatment device into a nostril of the patient to contact mucosal tissue of the upper airway with a treatment surface of the treatment device. Step 434 may be performed without piercing the mucosal tissue. The goal of the navigation may be to place the treatment element in contact with the treatment site.

During navigation, the clinician may perform one or more tests to determine whether proper contact with the treatment site has been made. In some embodiments, the clinician may activate one or more pairs of electrodes. Based on measured results, the clinician may determine that proper contact has not been achieved because an energy pathway could not be made between one or more pairs of electrodes and/or that one or more measured electrical parameters (e.g., impedance, voltage, current, temperature, etc.) is outside of a desired range. In some embodiments, the clinician may attempt to apply pressure to the treatment site with the treatment portion and determine by feel whether proper contact has been made. In some embodiments, clinician may take a reading using a thermocouple or an electrode to determine whether proper contact has been made. Based on the one or more tests, the clinician may determine that proper contact has been made between the treatment portion and the treatment site. During navigation, a navigation system may be used to position the device based on previously obtained images.

Energy can then be delivered to the treatment site in step 436. As is shown in FIG. 27, the physician may then move the treatment portion to another treatment site and repeat the steps of confirming contact and delivering energy. For example, as shown in FIG. 27, the treatment sites include a first target location 60, a second target location 62 and a third target location 64, all of which fall along the path of a posterior nasal nerve PNN. Additional treatments may optionally be provided in the same general area and/or in different areas of the nasal cavity. In certain circumstances, the clinician may determine that proper contact has not been made and the physician can then either re-orient the treatment portion or move it to another treatment site.

Once the treatment is complete, the treatment device may be removed from the nasal cavity in step 438.

The steps above and much of the disclosure is described with respect to the nose and nasal cavity, but it will be understood that the systems, devices, and methods described herein can be used in other areas of the body, including but not limited to the mouth, including the soft palate and the base of the tongue, the throat, including nerves in the throat, and the ear canal.

The systems and methods of the present disclosure can be used to treat various conditions inside or outside of the nasal cavity. Some of the conditions that may be treated by the embodiments described herein include, but are not limited to, rhinitis, asthma, COPD, emphysema, airway inflammation, nasal obstruction and/or congestion, eye inflammation and allergic conjunctivitis, chronic cough, post nasal drip, allergies, nasal polyps, sinusitis, turbinate enlargement, migraine headaches, other headaches (e.g., cluster headaches, nasal contact point headaches, etc.), tinnitus, dizziness, vertigo, dry eye, excessive tearing, empty nose syndrome, pain (e.g., facial nerve pain, trigeminal neuralgia, complex regional pain syndrome, etc.), sleep apnea, snoring, disordered breathing during sleep, anxiety, mood disorders, middle ear conditions (e.g., otitis media), Eustachian tube dysfunction, herpes zoster, paroxysmal hemicranias, cancer of the head or neck, and reduction of chemical mediators that lead to any of the above-listed conditions. It should be noted that many of these conditions may be co-morbid with one another, and so a treatment of one condition may alleviate one or more other conditions.

By way of non-limiting example, in some embodiments, the systems and methods of the present disclosure can be used to treat or alleviate rhinitis, which can be generally described as the inflammation of the nasal mucosa due to allergic or non-allergic triggers, which leads to excessive mucus production in the nasal cavity. This increased mucus can cause runny nose (rhinorrhea), post nasal drip syndrome (PNDS), upper airway cough syndrome (UACS), congestion, itching, and sneezing symptoms.

In some embodiments, the systems and methods of the present disclosure can be used to decrease mucus production in the upper airways. Mucus hypersecretion often occurs when a mucus producing cell displays rapid mucus secretion behavior. In some embodiments, to prevent this, properties of the cell can be modified to inactivate or retard the cell so as to halt or impede the release of mucins into the airway. In some embodiments, modifying the properties of the mucus producing cells may include reducing the rate at which cells produce mucus. In some embodiments, modifying the properties of mucus producing cells may include deactivating some or all mucus producing cells in a region. In some embodiments, the cells can be modified by being locally destroyed and can be replaced with healthier mucosal tissue, which may mean fewer mucous glands, fewer mucus producing cells, less inflammation, or more normal nerve function. While some embodiments may change a property of a tissue, such as an ability of a tissue to produce mucus, other embodiments may change a tissue from one type to another type. One example of this latter change is changing mucosal tissue containing goblet cells to mucosa with fewer goblet cells or scar tissue. These various embodiments may be used to reduce movement of mucus, reduce the amount of mucus produced, reduce frequency of mucus production, change the mucus viscosity/consistency, and/or change the path of mucus flow. In some embodiments, the systems and methods of the present disclosure can be used to apply energy to stimulate or disrupt various nasal nerves (as described below) to decrease mucus production in the upper airways.

By way of another non-limiting example, in some embodiments, the systems and methods of the present disclosure can be used to treat or alleviate migraine or headaches, such as cluster headaches, nasal contact point headaches, tension headaches, sinus headaches, or similar conditions. In some embodiments, the systems and methods of the present disclosure can be used to apply energy to stimulate or disrupt various nasal nerves (as described below) to alleviate migraines or headaches.

By way of another non-limiting example, in some embodiments, the systems and methods of the present disclosure can be used to treat or alleviate sleep apnea, disordered breathing during sleep other sleep disorders In some embodiments, tissues of the soft palate in the mouth can be reshaped, strengthened, stiffened or otherwise altered, including but not limited to skin, muscle, mucosa, submucosa and/or cartilage of the soft palate. This change in the soft palate can prevent collapse of the soft palate or vibration of the soft palate during nighttime breathing and thus prevent or at least reduce sleep apnea and/or snoring. One example of this is reducing nasal congestion through treatment of the nasal nerves leading to better breathing during sleep. This also reduces the negative pressure generated in the nasal cavity on inspiration leading to less airway collapse.

By way of another non-limiting example, in some embodiments, the systems and methods of the present disclosure can be used to treat or alleviate nasal airway obstruction, or to increase airflow, by, for example, modifying or reshaping various structures of the nasal cavity, such as the nasal valve area, turbinates, nasal wall, or similar tissue in the nasal cavity. For example, this can be used for improving breathing by decreasing airflow resistance or perceived airflow resistance at or near a site of an internal or external nasal valve. While, in some instances, nasal dysfunction can lead to poor airflow, nasal breathing can also be improved in people with normal breathing and/or normal nasal anatomy by decreasing nasal airflow resistance in the nasal valve and associated nasal anatomy. Remodeling or changing the structure of the nasal valve can improve nasal airflow in people with inadequate nasal airflow resulting from causes other than nasal valve dysfunction, such as a deviated septum, enlarged turbinates, mucosal swelling, and/or mucus overproduction.

By way of another non-limiting example, in some embodiments, the systems and methods of the present disclosure can be used for mucosal resurfacing or remodeling by delivering stimulus to mucosal tissue. A stimulus can be applied to cause local destruction of the mucosal layer and replacement with healthier mucosal tissue, which may mean fewer mucous glands, fewer mucus producing cells, less inflammation, fewer inflammatory cells, or more normal nerve function.

In some embodiments, the systems and methods of the present disclosure can be used to effect mucosal remodeling through direct stimulus from an agent or a stimulus. The resurfacing of the mucosa replaces the diseased or chronically inflamed mucosal cells with healthy mucosa that has more normal function, such as producing less mucus or other fluids, being less prone to allergy, and having less nerve stimulation and/or less activity of inflammatory cells. In some embodiments, this can be through modification of the cell types that comprise the mucosal layers. In some embodiments, this can include removing nerve ending present in the mucosal layer.

In some embodiments, the systems and methods of the present disclosure can be used to apply energy to submucosal tissue, such as mucus cells and glands. In some embodiments, the energy is applied to the posterior region of the nasal cavity.

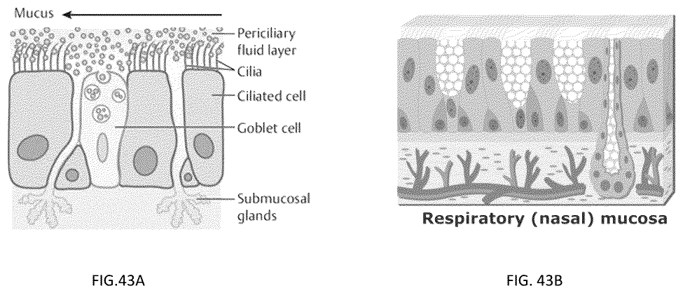

FIG. 43A and FIG. 43B show an exemplary cross-sectional view of the mucosa, according to one or more embodiments herein. In reference to FIGS. 43A and 43B, the stimulus can be delivered at a desired depth deeper than the thickness of the mucosa. The mucosal layer of the nasal cavity is between 0.5 mm and 5.0 mm thick, with an average thickness of 3.5 mm. In some embodiments, the energy is applied within submucosal tissue to destroy or vary a function of ciliated cells, goblet cells, or other epithelial cells of the nasal cavity, or mucous or serous glands.

In some embodiments, the systems and methods of the present disclosure can be used to apply a stimulus to neuromodulate certain nerves within the nasal cavity. In some embodiments, any nerve or combination of nerves in the nasal cavity can be treated to treat various conditions inside or outside of the nasal cavity. Generally, nasal neuromodulation may involve treating one or more nerves to change or disrupt their functions without completely stopping their function. At the same time, however, nasal neuromodulation may involve completely destroying the nerve tissue or stopping the function of one or more nerves while allowing one or more other nerves in the same area to maintain their function. Typically, the systems of the present disclosure will contact the mucosa overlying one or more of these one or more nerves, and energy will be delivered through the mucosa to neuromodulate the nerves. In some embodiments, the treatments of the present disclosure may involve disruption of parasympathetic and/or sensory nerve signals in one or more nerves (e.g., the posterior nasal nerve). In some embodiments, the destruction of submucosal tissue, as discussed above, may also change or disrupt the functions of the nerves in the same region (e.g., the posterior nasal region).

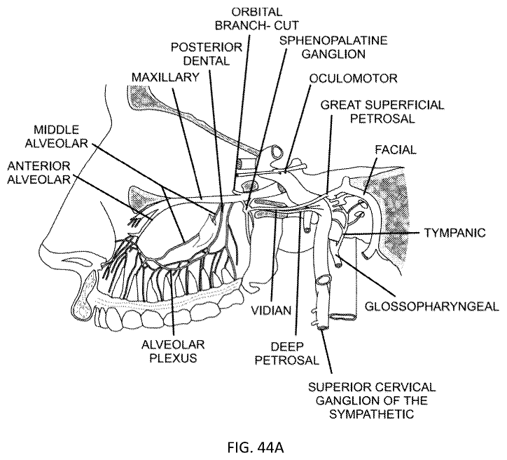

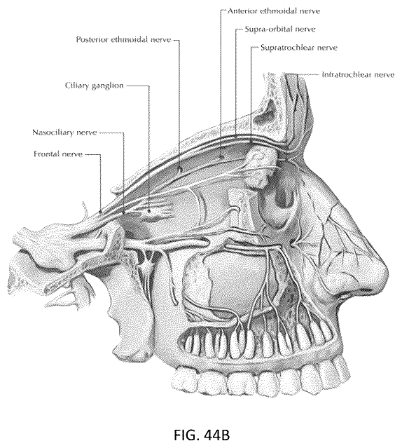

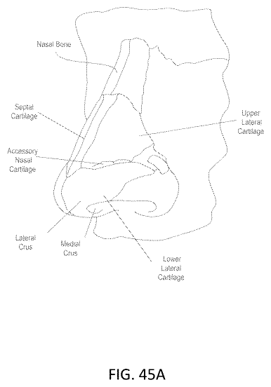

FIGS. 44A and 44B are sagittal views of a human nasal cavity, illustrating the lateral wall and nasal nerves that are related to the treatment of migraines and/or headaches. Referring to FIG. 44A, a sagittal cross-section of a human nasal cavity is shown. In this cross-sectional view, bone has been removed to illustrate the path of multiple different nerves, such as the vidian nerve and the sphenopalatine ganglion. According to various embodiments and examples described herein, any nerve or combination of nerves may be treated, such as but not limited, to the vidian nerve, vidian nerve branches, the sphenopalatine ganglion and/or any nerve branching from the sphenopalatine ganglion, such as the posterior nasal nerves (PNN), the posterolateral nasal nerves (PLNN), the posterior inferior lateral nasal nerves, and the posterior superior lateral nasal nerves (descending from the sphenopalatine ganglion in FIG. 44A but not labeled).

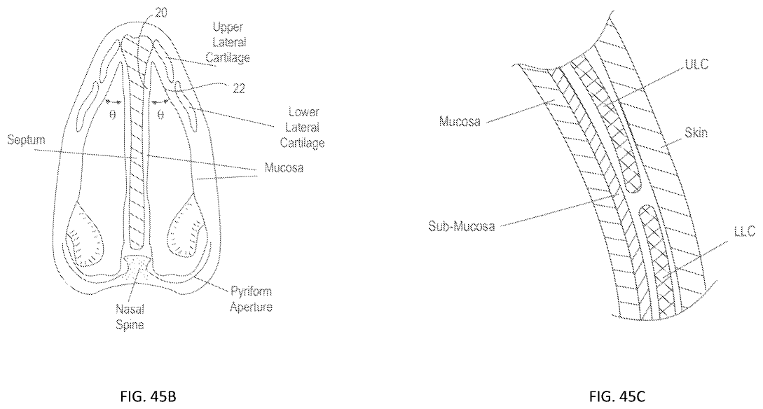

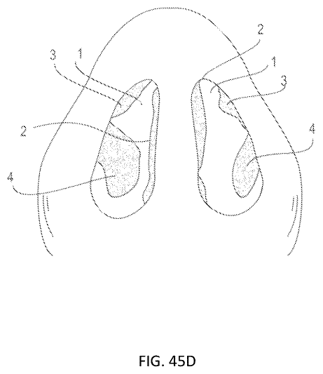

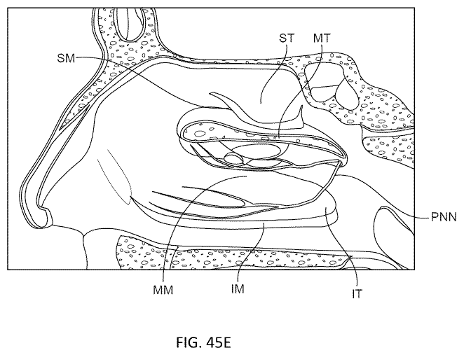

In reference to FIG. 45A, in some embodiments, nerves in the nasal cavity that can be treated include, but are not limited to, the frontal nerve, nasociliary nerve, ciliary ganglion, posterior nasal nerve, posterior ethmoidal nerve, supra-orbital nerve, infratrochlear nerve, olfactory nerve, trigeminal nerve, nasopalatine nerve, maxillary nerve, sphenopalatine ganglion, anterior nasal nerves, and the anterior ethmoidal nerve and its branches.

In some embodiments, the present disclosure includes neuromodulation of nerves in the posterior nasal region, such as posterior nasal nerves (PNN), the posterolateral nasal nerves (PLNN), the posterior inferior lateral nasal nerves, and the posterior superior lateral nasal nerves.

In some embodiments, the systems and methods of the present disclosure can be used to apply energy to reshape one or more tissues in the nasal cavity. FIGS. 45A, 45B, 45C, and 45D illustrate anatomical elements of a human nose. The lower lateral cartilage (LLC) includes an external component referred to as the lateral crus and an internal component referred to as the medial crus. The medial crus and septal nasal cartilage create a nasal septum that separates the left and right nostrils. Upper lateral cartilage lies between the lower lateral cartilages and the nasal bone. The left ULC is separated from the right ULC by the septal cartilage extending down the bridge of the nose. The opposing edges of the LLC and ULC may move relative to one another. Disposed between the opposing edges is an accessory nasal cartilage. The septal nasal cartilage and the ULC form an angle (THETA) called the nasal valve angle.

FIG. 45C illustrates a detailed cross-section of a segment of nose tissue in the area of the intersection of the ULC and the LLC. As shown in FIG. 45C and the detailed view of FIG. 45B, both inner and outer surfaces of the nasal cartilage are covered with soft tissue including mucosa, sub-mucosa and skin. FIG. 45D illustrates a view of the nose as seen from the nostrils. FIG. 45D depicts a nasal valve 1 shown between a septum 2 and a upper lateral cartilage 3. FIG. 45D also depicts the position of a turbinate 4.

In reference to FIG. 45E, three bony structures covered in mucosal tissue, called the superior, middle and inferior nasal turbinates, extend inwardly from each of the two lateral walls of the nasal cavity. FIG. 45E shows the right lateral wall of the nasal cavity, including the superior turbinate ST, superior meatus SM, a cut away middle turbinate MT, middle meatus MM, inferior turbinate IT and inferior meatus IM. The downwardly curved edge of each turbinate defines a passageway below each turbinate, known as a meatus. The inferior meatus is located beneath the inferior turbinate, the middle meatus is located beneath the middle turbinate, and the superior meatus is located beneath the superior turbinate.

In some embodiments, the devices of the present disclosure can be used to conformationally change the structure of one or more of these tissues of the nasal cavity.

For example, the systems of the present disclosure can be used to reshape the internal or external nasal valve anatomy to allow greater airflow without necessarily expanding the diameter of the nasal passage. In some embodiments, energy may be delivered into the nasal valve, submucosal tissue, lateral walls of the nasal cavity, nasal septum, the inferior anterior, and middle turbinates, or cartilage tissue to cause a conformational change and/or a change in the physical properties of the submucosal tissue or cartilage. For example, in some embodiments, energy may be applied to a targeted region of tissue adjacent to a nasal valve such that the tissue modification results in a tightening, shrinking, or enlarging of such targeted tissues resulting in a change of shape. For example, energy delivery may be accomplished by transferring the energy through the tissue covering the submucosa or cartilage such as the epithelium, mucosa, sub-mucosa, muscle, ligaments, tendon and/or skin.

In some embodiments, the tissue of the internal and/or external nasal valve and/or surrounding tissues may be strengthened or otherwise modified to resist a conformational change in response to the negative pressure of inspiration. In some embodiments, this strengthening may be performed by applying treatments selected to change mechanical or structural properties of the treated tissue. In some embodiments, such treatments may include the application of energy to selected regions of nasal valve and/or surrounding tissues.

Referring to FIG. 45E, a sagittal cross-section of a human nasal cavity is shown. In this cross-sectional view, the middle turbinate MT has been removed, to show the path of the posterior nasal nerve PNN. Also shown are the superior turbinate ST, superior meatus SM, middle meatus MM, inferior turbinate IT and inferior meatus IM. Although the systems, devices and methods described herein may be used to treat other nerves and other tissues in the nasal cavity, the PNN is one of the primary nerve tissue targets for treating chronic rhinitis, allergic rhinitis, post nasal drip, chronic cough, migraines, and/or other headache conditions. Another nerve, the ethmoid nerve (or “ethmoidal nerve”) and its branches are not shown in FIG. 45E, but any treatment device or method described herein may be used for treating the ethmoid nerve and/or its branches instead of, or in addition to, the PNN or any other nasal nerve(s). For the purposes of this application, the term “rhinitis” is used as an inclusive term to describe any one of, or a combination of, chronic rhinitis, allergic rhinitis, post nasal drip and/or chronic cough, as well as any symptoms that stem therefrom. In an actual patient, the PNN and other nasal nerves lie under the nasal mucosa and thus are not visible or on the surface, as they are in FIG. 45E. This anatomical drawing is provided for illustration purposes only.

In one aspect of the present disclosure, a system for treating nasal airway tissue includes a console and a stylus. The console includes a housing, a pulsed electric field generator in the housing, a computer processor in the housing, and an outlet on the housing. The stylus includes a handle, a power cord connected to a first end of the handle (the power cord including a connector at an opposite end for connecting to the outlet), a shaft extending from a second end of the handle, and a distal tip extending from a distal end of the shaft. The distal tip includes a treatment surface and multiple electrodes on the treatment surface configured to deliver pulsed electric field energy provided by the pulsed electric field generator.

In some embodiments, the shaft of the stylus is malleable and has a width of 4 millimeters to 5 millimeters. In some embodiments, the multiple electrodes comprise two rows of protruding, non-piercing electrodes. In some embodiments, the treatment surface is convex. In some embodiments, the shaft of the stylus has a length of at least 3.75 inches.

Optionally, the system may also include a power cord coupled with the console, and a foot pedal attachable to the console for activating the console to supply the pulsed electric field energy to the stylus. The system may also include an on/off button on the stylus for activating the console to supply the electrical energy to the stylus. In various embodiments, the pulsed electric field generator is configured to provide exponentially decaying, square wave, bipolar, monophasic, biphasic, and/or oscillatory pulses.

In another aspect of a present disclosure, a method for treating a nasal airway involves activating a pulsed electric field generator console attached to a stylus, advancing a distal tip of the stylus into a nostril of the patient, and contacting nasal mucosa lining the nasal airway with a treatment surface of the distal tip. The method then involves delivering pulsed electric field energy from at least a first electrode on the treatment surface to at least a second electrode on the treatment surface, to treat a tissue underlying the nasal mucosa, and removing the distal tip of the stylus from the nostril.

In some embodiments, the method may further involve moving the distal tip to multiple additional locations within the nasal airway and delivering the pulsed electric field energy to nasal airway tissue at the multiple additional locations. In some embodiments, the tissue comprises at least one nasal nerve. For example, the nasal nerve may be, but is not limited to, a posterior nasal nerve or an anterior ethmoid nerve.

In some embodiments, the method may include sensing a temperature of the nasal mucosa with a temperature sensing member on the treatment surface of the distal tip. Such a method may optionally further involve automatically shutting off delivery of the pulsed electric field energy from the console to the stylus if the sensed temperature is above a predefined acceptable maximum temperature. Alternatively or additionally, the method may include sensing an impedance in tissue between the first electrode and the second electrode with an impedance sensing member. Such a method may also include automatically shutting off delivery of the pulsed electric field energy from the console to the stylus if the sensed impedance is outside a predefined acceptable impedance range.

Optionally, the method may further involve bending the shaft before inserting the distal tip into the nostril. In some embodiments, the first electrode comprises a first row of electrodes, and the second electrode comprises a second row of electrodes. The method may also involve injecting an anesthetic fluid into the nasal mucosa before advancing the distal tip of the stylus into the nostril, to enhance conduction of the delivered electrical energy through the mucosal tissue.

In some embodiments, delivering the pulsed electric field energy ablates the tissue underlying the nasal mucosa. In some embodiments, the tissue comprises nerve tissue, and the method further involves delivering the pulsed electric field energy to an additional tissue, such as but not limited to an inferior turbinate, a middle turbinate, a superior turbinate, a nasal septum, and a septal swell body.

Medical devices that use energy to treat tissue in the body are used for a wide variety of procedures, to treat many different conditions and injuries. For example, in some embodiments, an energy delivery device is inserted into a patient's nostril to deliver energy (and sometimes mechanical force) to tissue in the nose. This type of energy delivery treatment may be used to reshape cartilage and/or other structures in the nose and/or to change other properties of tissues, such as nerves underlying the nasal mucosa lining the walls of the nasal cavity. The treatments may address any of a wide variety of conditions, just two examples of which are nasal valve insufficiency (which may cause difficulty breathing through the nose) and chronic or allergic rhinitis. Examples of nasal tissue treatment devices, systems and methods patented by the applicant include U.S. Pat. Nos. 8,936,594; 9,486,278; 8,986,301; 9,027,597; 9,179,964; 9,452,010; 9,415,194; 9,179,967; 9,788,886; 9,801,752; 9,433,463; 10,335,221; 9,943,361; 9,687,296; 10,398,489; 9,237,924; 9,526,571; 9,913,682; 10,028,780; 10,265,115; 10,485,603; 10,376,300; 10,456,186; 10,456,185; 9,888,957; and 10,470,814, and U.S. patent application Ser. No. 16/668,678, all of which are hereby incorporated by reference in this application, and all of which may be referred to collectively herein as the “Incorporated References.”

Although this application is believed to be complete and accurate, any suitable changes may be made to any of the described embodiments and features described above, without departing from the scope of the invention.

We claim:

1. A system for treating tissue in a nasal airway, the system comprising:
a console comprising a pulsed electric field generator configured to generate pulsed electric field energy in the form of high frequency alternating pulses of energy delivered as a pulse train, the pulsed electric field generator comprising a plurality of output channels; and
a treatment device comprising an elongate shaft having a treatment surface on a distal end thereof, the treatment surface comprising a plurality of electrode pairs configured to deliver the pulsed electric field energy provided by the pulsed electric field generator to a target tissue in the nasal airway,
wherein each of the plurality of output channels is connected with a subset of the plurality of electrode pairs to provide pulsed electric field energy to the plurality of electrode pairs.

2. The system of claim 1, wherein the pulsed electric field generator is configured to deliver pulsed field ablation energy to the plurality of electrode pairs.

3. The system of claim 1, wherein the pulsed electric field generator is configured to deliver pulsed radiofrequency energy to the plurality of electrode pairs.

4. The system of claim 1, wherein the pulse train is configured to be delivered in repeated intervals to create a string of pulse trains with an interval of time between pulse trains to allow for tissue recovery between each pulse train.

5. The system of claim 1, wherein the plurality of output channels is configured to connect with the plurality of electrode pairs to provide independent pulsed electric energy from each of the plurality of output channels to each of subsets of the plurality of electrode pairs.

6. The system of claim 1, further comprising at least one sensor configured to measure a temperature of the target tissue.

7. The system of claim 1, wherein the shaft of the treatment device is malleable and has a width of 4 millimeters to 5 millimeters.

8. The system of claim 1, wherein the plurality of electrode pairs comprises at least two rows of electrodes pairs.

9. The system of claim 1, wherein the pulsed electric field generator is configured to provide at least one of exponentially decaying, square wave, bipolar, or oscillatory pulses.

10. A system for treating nasal airway tissue, the system comprising:
a pulsed electric field generator configured to generate pulsed electric field energy in the form of alternating pulses of energy delivered as a pulse train, the pulsed electric field generator comprising a plurality of output channels; and
a treatment device comprising an elongate shaft having a treatment surface on a distal end thereof, the treatment surface comprising a first row of electrodes comprising a first plurality of electrode pairs and a second row of electrodes comprising a second plurality of electrode pairs configured to deliver pulsed electric field energy provided by the pulsed electric field generator to a target tissue in the nasal airway;
wherein each of the plurality of output channels is connected with a subset of the first and second plurality of electrode pairs to provide pulsed electric field energy to the plurality of electrode pairs.

11. A method for treating a nasal airway, the method comprising:
activating a pulsed electric field generator console attached to a treatment device to generate pulsed electric field energy in the form of high frequency alternating pulses of energy delivered as a pulse train;
advancing a distal tip of the treatment device into the nasal airway of a patient;
contacting a nasal mucosa lining the nasal airway with a treatment surface at a distal end of the treatment device;
delivering the pulsed electric field energy from at least a first electrode on the treatment surface to at least a second electrode on the treatment surface, to treat a tissue underlying the nasal mucosa lining; and
removing the distal tip of the treatment device from the nasal airway.

12. The method of claim 11, further comprising moving the distal tip to multiple additional locations within the nasal airway; and delivering the pulsed electric field energy to nasal airway tissue at the multiple additional locations.

13. The method of claim 11, wherein the tissue comprises at least one nasal nerve selected from the group consisting of a posterior nasal nerve and an anterior ethmoid nerve.

14. The method of claim 11, further comprising sensing a temperature of the nasal mucosa lining with a temperature sensing member on the treatment surface of the distal tip.

15. The method of claim 14, further comprising automatically shutting off delivery of the pulsed electric field energy from the pulsed electric field generator if the temperature is above a predefined acceptable maximum temperature.

16. The method of claim 11, further comprising:
sensing an impedance in the tissue between the first electrode and the second electrode with an impedance sensing member; and
automatically shutting off delivery of the pulsed electric field energy from the pulsed electric field generator console to the treatment device if the impedance is outside a predefined acceptable impedance range.

17. The method of claim 11, wherein the at least the first electrode comprises a first row of electrodes, and wherein the at least the second electrode comprises a second row of electrodes.

18. The method of claim 11, further comprising injecting an anesthetic fluid into the nasal mucosa lining before advancing the distal tip of the treatment device into the nasal airway, to enhance conduction of the pulsed electric field energy through the tissue.

19. The method of claim 11, wherein delivering the pulsed electric field energy ablates the tissue underlying the nasal mucosa lining.

20. The method of claim 19, wherein the tissue comprises nerve tissue, and wherein the method further comprises delivering the pulsed electric field energy to an additional tissue selected from the group consisting of an inferior turbinate, a middle turbinate, a superior turbinate, a nasal septum, and a septal swell body.
